(19) United States
(12) Patent Application Publication
(10) Pub. No.: US 2025/0019451 A1
(43) Pub. Date: Jan. 16, 2025

(54) ANTI-CCR8 ANTIBODIES AND USES THEREOF

(71) Applicant: HIFIBIO, INC., Cambridge, MA (US)

(72) Inventors: Julie PRIGENT, Maisons-Alfort (FR); Nicola Arturo Aldo BELTRAMINELLI, Villeurbanne (FR); Sami ELLOUZE, Chatenay-Malabry (FR); Francisco ADRIAN, Cambridge, MA (US); Liang SCHWEIZER, Cambridge, MA (US); Yun-Yueh LU, Shanghai (CN); Ross Bane FULTON, Southborough, MA (US)

(73) Assignee: HIFIBIO, INC., Cambridge, MA (US)

(21) Appl. No.: 18/685,068

(22) PCT Filed: Aug. 19, 2022
(86) PCT No.: PCT/CN2022/113739

§ 371 Date: Feb. 20, 2024

(30) Foreign Application Priority Data
Aug. 20, 2021 (WO) PCT/CN2021/113913

(51) Int. Cl. C07K 16/28; A61K 39/00; A61P 35/00; G01N 33/574

(52) U.S. Cl. CPC C07K 16/2866; A61P 35/00; G01N 33/57484; A61K 2039/505; C07K 2317/24; C07K 2317/33; C07K 2317/41; C07K 2317/52; C07K 2317/732; C07K 2317/92; G01N 2333/7158

(57) ABSTRACT

Anti-CCR8 antibodies and antigen-binding fragments thereof are described herein. Also described herein are nucleic acids encoding the anti-CCR8 antibodies and antigen-binding fragments thereof, compositions comprising the anti-CCR8 antibodies and antigen-binding fragments thereof, and methods of producing and using the anti-CCR8 antibodies and antigen-binding fragments thereof for treating or preventing cancer in a subject in need thereof.

### CROSS-REFERENCE TO RELATED APPLICATIONS

This application is the United States national phase under 35 U.S.C. § 371 of International Patent Application No. PCT/CN2022/113739 filed Aug. 19, 2022, which claims benefit of priority under 35 U.S.C. § 119 (c) to International Patent Application No. PCT/CN2021/113913 filed Aug. 20, 2021, now withdrawn. The disclosure of the prior applications is considered part of and is herein incorporated by reference in the disclosure of this application in its entirety.

### FIELD OF THE INVENTION

This invention relates to isolated anti-chemokine (C—C motif) receptor 8 (CCR8) monoclonal antibodies or antigen-binding fragments thereof, nucleic acids and expression vectors encoding the antibodies, recombinant cells containing the vectors, and compositions comprising the antibodies. Methods of making the antibodies, and methods of using the antibodies to treat diseases including cancer and/or associated complications are also provided.

### REFERENCE TO SEQUENCE LISTING SUBMITTED ELECTRONICALLY

This application contains a sequence listing, which is submitted electronically via Patent Center as an XML sequence listing with a file name G1241US00_148640-004212.xml and a creation date of Sep. 30, 2024 and having a size of 84 kb. The sequence listing submitted via Patent Center is part of the specification and is herein incorporated by reference in its entirety.

### BACKGROUND OF THE INVENTION

CCR8 is a chemokine receptor mediating cell migration under CCL1 or CCL18 gradient (Islam et al., JEM 210 (10): 1889-1898 (2013)). Recently, CCR8 was identified as a highly specific cell-surface marker of tumor infiltrating regulatory T cells (TITRs) in human cancers with considerably higher expression in Tregs residing within tumors as compared to Tregs in circulation, and no or very low expression on other T-cell population (cytotoxic T-cells or effector T-cell, respectively). Furthermore, CCR8 is predominantly expressed on high immunosuppressive Tregs that expressed FoxP3high, CD25high, TIGIT+, LAG3+ and release high IL-10 and TGF-β. Depleting CCR8+Tregs would decrease immunosuppressives cytokines and modulate a pro-tumoral microenvironment to restore anti-tumor immunity. Interestingly, in patients with breast or pancreatic cancer, high CCR8+ Treg numbers correlated with more advanced stages of the disease and decreased probably of overall survival. Therefore, CCR8 is an ideal target for cancer immunotherapies to treat and potentially cure CCR8-positive cancers.

### BRIEF SUMMARY OF THE INVENTION

In one general aspect, the invention relates to isolated monoclonal antibodies or antigen-binding fragments thereof that specifically bind chemokine (C—C motif) receptor 8 (CCR8).

Provided are isolated monoclonal antibodies or antigen-binding fragments thereof comprising a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 1, 2, 3, 4, 5, and 6, respectively;
(2) SEQ ID NOs: 13, 2, 14, 4, 28, and 6, respectively;
(3) SEQ ID NOs: 13, 2, 15, 4, 5, and 6, respectively;
(4) SEQ ID NOs: 16, 17, 18, 29, 30, and 6, respectively;
(5) SEQ ID NOs: 19, 20, 21, 4, 5, and 6, respectively;
(6) SEQ ID NOs: 22, 23, 24, 31, 5, and 32, respectively; or
(7) SEQ ID NOs: 25, 26, 27, 33, 34, and 35, respectively;
   wherein the antibody or antigen-binding fragment thereof specifically binds chemokine (C—C motif) receptor 8 (CCR8), preferably human CCR8.

In certain embodiments, the isolated monoclonal antibody or antigen-binding fragment thereof comprises a heavy chain variable region having a polypeptide sequence at least 90%, at least 95%, at least 96%, at least 97%, at least 98%, or at least 99% identical to SEQ ID NO:7, 36, 38, 40, 42, 44, or 46, or a light chain variable region having a polypeptide sequence at least 90%, at least 95%, at least 96%, at least 97%, at least 98%, or at least 99% identical to SEQ ID NO:8, 37, 39, 41, 43, 45, or 47.

In certain embodiments, the isolated anti-CCR8 monoclonal antibody or antigen-binding fragment thereof comprises:
(1) a heavy chain variable region having the polypeptide sequence of SEQ ID NO:7, and a light chain variable region having the polypeptide sequence of SEQ ID NO:8;
(2) a heavy chain variable region having the polypeptide sequence of SEQ ID NO:36, and a light chain variable region having the polypeptide sequence of SEQ ID NO:37;
(3) a heavy chain variable region having the polypeptide sequence of SEQ ID NO:38, and a light chain variable region having the polypeptide sequence of SEQ ID NO:39;
(4) a heavy chain variable region having the polypeptide sequence of SEQ ID NO:40, and a light chain variable region having the polypeptide sequence of SEQ ID NO:41;
(5) a heavy chain variable region having the polypeptide sequence of SEQ ID NO:42, and a light chain variable region having the polypeptide sequence of SEQ ID NO:43;
(6) a heavy chain variable region having the polypeptide sequence of SEQ ID NO:44, and a light chain variable region having the polypeptide sequence of SEQ ID NO:45; or
(7) a heavy chain variable region having the polypeptide sequence of SEQ ID NO:46, and a light chain variable region having the polypeptide sequence of SEQ ID NO:47.

In certain embodiments, the isolated monoclonal antibody or antigen-binding fragment thereof binds to a CCR8 and is capable of inducing effector-mediated tumor cell lysis through antibody-dependent cellular cytotoxicity (ADCC), antibody-dependent cellular phagocytosis (ADCP), and/or mediating the activity of conjugated drugs; and/or forming a bispecific antibody with another monoclonal antibody or antigen-binding fragment thereof with cancer-killing effect.

In certain embodiments, the isolated monoclonal antibody or antigen-binding fragment thereof is chimeric or human or humanized.

In certain embodiments, the humanized monoclonal antibody or antigen-binding fragment thereof comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 1, 48, 49, 50, 51, and 6, respectively;
(2) SEQ ID NOs: 1, 2, 49, 4, 5, and 6, respectively;
(3) SEQ ID NOs: 1, 2, 49, 50, 51, and 6, respectively;
(4) SEQ ID NOs: 1, 2, 3, 4, 5, and 6, respectively; or
(5) SEQ ID NOs: 1, 48, 49, 50, 51, and 6, respectively.

In certain embodiments, the isolated monoclonal antibody or antigen-binding fragment thereof comprises a heavy chain variable region having a polypeptide sequence at least 90%, at least 95%, at least 96%, at least 97%, at least 98%, or at least 99% identical to SEQ ID NO:9, 52, 53, 54, 55, 56, 57, 58, 59, or 60, or a light chain variable region having a polypeptide sequence at least 90%, at least 95%, at least 96%, at least 97%, at least 98%, or at least 99% identical to SEQ ID NO: 10, 61, 62, or 63.

In certain embodiments, the isolated anti-CCR8 monoclonal antibody or antigen-binding fragment thereof comprises:
(1) a heavy chain variable region having the polypeptide sequence of SEQ ID NO:9, and a light chain variable region having the polypeptide sequence of SEQ ID NO:10;
(2) a heavy chain variable region having the polypeptide sequence of SEQ ID NO:52, and a light chain variable region having the polypeptide sequence of SEQ ID NO:63;
(3) a heavy chain variable region having the polypeptide sequence of SEQ ID NO:52, and a light chain variable region having the polypeptide sequence of SEQ ID NO:10;
(4) a heavy chain variable region having the polypeptide sequence of SEQ ID NO:53, and a light chain variable region having the polypeptide sequence of SEQ ID NO:10;
(5) a heavy chain variable region having the polypeptide sequence of SEQ ID NO:54, and a light chain variable region having the polypeptide sequence of SEQ ID NO:63; or
(6) a heavy chain variable region having the polypeptide sequence of SEQ ID NO:59, and a light chain variable region having the polypeptide sequence of SEQ ID NO:62.

In certain embodiments, the isolated monoclonal antibody or antigen-binding fragment thereof specifically binds cynomolgus CCR8.

Also provided are isolated bispecific antibodies or antigen-binding fragments thereof comprising the monoclonal antibodies or antigen-binding fragments thereof of the invention.

Also provided are isolated nucleic acids encoding the monoclonal antibodies or antigen-binding fragments thereof or bispecific antibodies or antigen-binding fragments thereof of the invention.

Also provided are vectors comprising the isolated nucleic acids encoding the monoclonal antibodies or antigen-binding fragments thereof or bispecific antibodies or antigen-binding fragments thereof of the invention.

Also provided are host cells comprising the vectors comprising the isolated nucleic acids encoding the monoclonal antibodies or antigen-binding fragments thereof or bispecific antibodies or antigen-binding fragments thereof of the invention.

In certain embodiments, provided is a pharmaceutical composition comprising an isolated monoclonal antibody or antigen-binding fragment thereof or an isolated bispecific antibody or antigen-binding fragment thereof of the invention and a pharmaceutically acceptable carrier.

Also provided are methods of specifically targeting C—C motif chemokine receptor 8 (CCR8) on a cancer cell surface in a subject in need thereof, comprising administering to the subject a pharmaceutical composition of the invention.

Also provided are methods of treating cancer in a subject in need thereof, comprising administering to the subject the pharmaceutical compositions of the invention. The cancer can, for example, be a solid tumor, preferably a solid tumor with infiltrating T cells, more preferably a solid tumor with infiltrating T reg cells, more preferably a solid tumor with highly suppressive T reg cells expressing CCR8, most preferably a solid tumor with infiltrating highly suppressive T reg cells overexpressing CCR8 for which natural killer (NK) cell infiltration has occurred. Examples of cancers can, for example, be selected from, but not limited to, lung cancer, head and neck cancer, esophageal cancer, stomach cancer, colorectal cancer, breast cancer, pancreatic cancer, ovarian cancer, bladder cancer, liver cancer, kidney cancer, and melanoma. In certain embodiments, the subject can, for example, comprise CCR8-expressing Treg cells.

Also provided are methods of producing a monoclonal antibody or antigen-binding fragment thereof or bispecific antibody or antigen-binding fragment thereof of the invention. The methods comprise culturing a cell comprising a nucleic acid encoding the monoclonal antibody or antigen-binding fragment thereof or bispecific antibody or antigen-binding fragment thereof under conditions to produce the monoclonal antibody or antigen-binding fragment thereof or bispecific antibody or antigen-binding fragment thereof, and recovering the monoclonal antibody or antigen-binding fragment thereof or bispecific antibody or antigen-binding fragment thereof from the cell or culture.

Also provided are methods of producing a pharmaceutical composition comprising a monoclonal antibody or antigen-binding fragment thereof or bispecific antibody or antigen-binding fragment thereof of the invention. The methods comprise combining the monoclonal antibody or antigen-binding fragment thereof or bispecific antibody or antigen-binding fragment thereof with a pharmaceutically acceptable carrier to obtain the pharmaceutical composition.

Also provided are methods of determining the level of a CCR8 in a subject. The methods comprise (a) obtaining a sample from the subject; (b) contacting the sample with an anti-CCR8 monoclonal antibody or antigen-binding fragment thereof of the invention; and (c) determining the level of CCR8 in the subject. In certain embodiments, the sample is a tissue sample. The tissue sample can, for example, be a cancer tissue sample. In certain embodiments, the sample is a blood sample. In certain embodiments, the sample comprise Treg cells.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1C show that anti-CCR8 monoclonal antibodies bind specifically to CCR8 expressing cells with a sub-nM EC₅₀. FIG. 1A shows binding of parental antibodies on CCR8.CHO cells. FIG. 1B shows binding of parental antibodies on parental cells. FIG. 1C is a table summarizing the EC₅₀ measured in FIGS. 1A and 1B.

FIG. 2 shows that anti-CCR8 monoclonal antibodies recognize the N-terminal epitope, as well as the hCCR8 loop 1, which is involved in protein conformation, and do not recognize hCCR4. Top: drawing of the chimeric constructs (black: hCCR8, grey: hCCR4); bottom: binding profiles associated with the constructs.

FIGS. 3A-3C show that anti-CCR8 monoclonal antibodies bind to human CCR8 protein and can cross react with the mouse and/or cynomolgus CCR8 protein. FIG. 3A shows anti-CCR8 monoclonal antibodies binding to human, mouse and cynomolgus CCR8 protein.

FIG. 3B shows the EC50 of HFB11-21 and HFB11-19. FIG. 3C shows the EC50 of HFB11-21 and HFB11-2.

FIGS. 4A-4B show different CCL1 blocking profiles for anti-CCR8 monoclonal antibodies (FIG. 4A) and the associated IC₅₀ table (FIG. 4B). The cyno-cross-reactive antibodies do not block hCCL1, rather the strong hCCR8 binding antibodies block hCCL1.

FIGS. 5A-5G show that anti-CCR8 monoclonal antibodies inhibit intracellular Ca²⁺ flux. FIG. 5A shows the Ca²⁺ changes in the presence of buffer. FIG. 5B shows the Ca²⁺ changes in the presence of 1 nM CCL1. FIG. 5C shows the Ca²⁺ changes in the presence of 1 nM CCL1+HFB11-3. FIG. 5D shows the Ca²⁺ changes in the presence of 1 nM CCL1+HFB11-10.

FIG. 5E shows the Ca²⁺ changes in the presence of buffer+10 nM CCL1. FIG. 5F shows the Ca²⁺ changes in the presence of 10 nM CCL1+HFB11-3. FIG. 5G shows the Ca²⁺ changes in the presence of 10 nM CCL1+HFB11-10.

FIGS. 6A-6B show that anti-CCR8 antibodies potently engage CD16 in an ADCC reporter bioassay and mediate ADCC through CD16-engineered cells. FIG. 6A shows the RLU signal after incubation for 5 minutes with the luciferase substrate. FIG. 6B shows a table of EC₅₀ and Emax values for all antibodies.

FIGS. 7A-7B show that the humanized anti-CCR8 antibodies in ADCC-enhanced format bind specifically to CCR8 expressing cells. FIG. 7A shows the MFI signal. FIG. 7B shows a table of EC₅₀ values for all antibodies.

FIGS. 8A-8B show that the humanized anti-CCR8 antibodies block CCL1 binding to CCR8 expressing cells. FIG. 8A shows the percent CCL1 blocking. FIG. 8B shows a table of IC₅₀ values for all antibodies.

FIG. 9 shows that the humanized HFB11-10 Hz37 anti-CCR8 antibody engages both CD16 F and V variants in an ADCC reporter bioassay.

FIGS. 10A-10B show that the humanized anti-CCR8 antibodies mediate ADCC on CCR8 expressing cells. FIG. 10A shows the percent specific lysis. FIG. 10B shows a table of EC₅₀ values for all antibodies.

FIGS. 11A-11G show the in vitro characterization of anti-CCR8 mAb HFB101110. FIG. 11A shows the binding of HFB101110 to high-copy (CHOK1-hCCR8, ˜30,000 receptors/cell, left) or low-copy (M300.19-hCCR8, ˜2,000 receptors/cell, right) cells as measured by flow cytometry.

FIG. 11B shows ADCC activity of HFB101110 against M300.19-hCCR8 cells. FIG. 11C shows the binding of HFB101110 to the related chemokine receptor CCR4 was evaluated by flow cytometry. FIG. 11D shows domain swapping experiments to identify the region of CCR8 recognized by HFB101110. FIG. 11E shows the blockade of hCCL1 binding to hCCR8 by HFB101110, measured by flow cytometry. FIG. 11F shows HFB101110-mediated blockade of chemotaxis of CCR8+ cells induced by recombinant hCCL1, as measured by a transwell migration assay. FIG. 11G shows HFB101110-mediated blockade of calcium flux induced by the addition of hCCL1 to CCR8+ cells.

FIG. 12 shows that that humanized HFB11-10 Hz37 anti-CCR8 antibody mediates ADCP of CCR8+ expressing cells.

FIGS. 13A-13C show that the humanized anti-CCR8 antibody mediates antitumor activity to MC38 cells in hCCR8-KI mice. FIG. 13A shows tumor volumes after treatment with MG053 isotype. FIG. 13B shows tumor volumes after treatment with Hz varlant. FIG. 13C shows a comparison of tumor volumes after treatment with MG053 isotype or Hz varlant.

FIGS. 14A-14F show that the humanized anti-CCR8 antibody therapy reprograms the tumor microenvironment in vivo. FIG. 14A shows the percent Tregs among CD4+ after treatment with an isotype or with HFB11-10 Hz37. FIG. 14B shows the percent CD4+T effector among CD4+ after treatment with an isotype or with HFB11-10 Hz37. FIG. 14C shows the percent CD8+ among CD3+ after treatment with an isotype or with HFB11-10 Hz37. FIG. 14D shows the CD8+/Treg ratio after treatment with an isotype or with HFB11-10 Hz37. FIG. 14E shows the percent natural killer among CD45+ after treatment with an isotype or with HFB11-10 Hz37. FIG. 14F shows the percent CCR8+ among Treg after treatment with an isotype or with HFB11-10 Hz37.

FIGS. 15A-15B shows CCR8 expression in the T-cell population from human primary tumors from renal cell cancer (RCC) patients and from lung cancer patients. Each point represents data from an individual patient (FIG. 15A). FIG. 15B shows a histogram overlaying CCR8 expression in CD8+ (CD8+CD3+), Teff (FoxP3-CD4+CD3+), and Treg (FoxP3+CD4+CD3+) from TILs of an RCC patient.

FIG. 16 shows CCR8 expression in the T-cell population from the circulation in healthy and malignant PBMCs.

FIGS. 17A-17E show ex vivo ADCC activity mediated by the humanized HFB11-10 antibody on primary human TILs from RCC patients (n=13). FIG. 17A shows a schematic of sample collection and ADCC assay. FIG. 17B shows depletion of different T cell subsets. Each point represents data from an individual patient. FIG. 17C shows a dot plot showing Treg population gated as CD3+ CD4+ Foxp3+ TIGIT+ cells from isotype group versus treated group with HFB11-10 Hz antibody at 50 nM in the TILs from a representative RCC patient. FIG. 17D shows a dose-response of Treg-depleting activity of HFB101110. FIG. 17E shows depletion of Tregs in ADCC assay as a function of amount of exogenous NK cells added.

FIGS. 18A-18C show safety and pharmacokinetics study. FIG. 18A shows a schematic of single-dose PK study in cynomolgus monkeys. FIG. 18B shows serum PK profile of HFB101110 in cynomolgus monkeys. FIG. 18C shows in vitro cytokine release study from human PBMCs using a soluble antibody format.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right,” “left,” “lower,” and “upper” designate directions in the drawings to which reference is made.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., anti-CCR8 antibodies and polynucleotides that encode them, CCR8 polypeptides and CCR8 polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 1981; 2:482, by the homology alignment algorithm of Needleman & Wunsch, J. Mol. Biol. 1970; 48:443, by the search for similarity method of Pearson & Lipman, Proc. Nat'l. Acad. Sci. USA 1988; 85:2444, by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., 1995 Supplement (Ausubel)).

Examples of algorithms that are suitable for determining percent sequence identity and sequence similarity are the BLAST and BLAST 2.0 algorithms, which are described in Altschul et al., J. Mol. Biol. 1990; 215:403-410 and Altschul et al., Nucleic Acids Res. 1997; 25:3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 1989; 89:10915).

In addition to calculating percent sequence identity, the BLAST algorithm also performs a statistical analysis of the similarity between two sequences (see, e.g., Karlin & Altschul, Proc. Nat'l. Acad. Sci. USA 1993; 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

### Antibodies

The invention generally relates to isolated anti-chemokine (C—C motif) receptor 8 antibodies, nucleic acids and expression vectors encoding the antibodies, recombinant cells containing the vectors, recombinant cells expressing the antibodies, and compositions comprising the antibodies. Methods of making the antibodies, and methods of using the antibodies to treat diseases, such as cancer, are also disclosed. The antibodies of the invention possess one or more desirable functional properties, including, but not limited to, high-affinity binding to CCR8, high specificity to CCR8, the ability to stimulate antibody-dependent cellular phagocytosis (ADCP) and/or antibody-dependent cellular-mediated cytotoxicity (ADCC) against cells expressing CCR8, and the ability to inhibit tumor growth in subjects and animal models when administered alone or in combination with other anti-cancer therapies.

In a general aspect, the invention relates to isolated monoclonal antibodies or antigen-binding fragments thereof that bind chemokine (C—C motif) receptor 8 (CCR8).

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to CCR8 is substantially free of antibodies that do not bind to CCR8). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that may be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′).

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” and/or “humanized antigen binding domain” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multi-specific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., different proteins (or different subunits of a multimeric protein). In an embodiment, a multi-specific antibody comprises a third, fourth, or fifth immunoglobulin variable domain. In an embodiment, a multi-specific antibody is a bispecific antibody molecule, a tri-specific antibody molecule, or a tetra-specific antibody molecule.

As used herein, the term “bispecific antibody” refers to a multi-specific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on CCR8 and the second epitope is located on PD-1, PD-L1, CTLA-4, EGFR, HER-2, CD19, CD20, CD33, CD3, and/or other tumor associated immune suppressors or surface antigens.

As used herein, an antibody that “specifically binds to CCR8” refers to an antibody and/or antigen binding domain that binds to CCR8, preferably human CCR8, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. In certain embodiments, the antibody and/or antigen-binding domain binds to cynomolgus CCR8. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system.

The smaller the value of the KD of an antibody, the higher affinity that the antibody binds to a target antigen.

As used herein the term “IC₅₀” refers to the half maximal inhibitory concentration of a monoclonal or bispecific antibody or antigen-binding fragment thereof of the invention. IC₅₀ is a measure of the potency of the monoclonal or bispecific antibody or antigen-binding fragment thereof of the invention for inhibiting the binding of CCL1 to CCR8 or inhibiting the function of CCR8 in a cell. In certain embodiments, the monoclonal antibody or antigen-binding fragment thereof or the bispecific antibody or antigen-binding fragment thereof has a KD of less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M.

As used herein the term “EC₅₀” refers to the half maximal effective concentration of a monoclonal or bispecific antibody or antigen-binding fragment thereof of the invention. EC₅₀ refers to the concentration of a monoclonal or bispecific antibody or antigen-binding fragment thereof for inducing a biological response (i.e., cell death) halfway between the baseline and maximum over a specified exposure time. In certain embodiments, the monoclonal antibody or antigen-binding fragment thereof or the bispecific antibody or antigen-binding fragment thereof has an EC₅₀ of less than about 1 μM, about 1000 nM to about 100 nM, about 100 nM to about 10 nM, about 10 nM to about 1 nM, about 1000 pM to about 500 pM, about 500 pM to about 200 pM, less than about 200 pM, about 200 pM to about 150 pM, about 200 pM to about 100 pM, about 100 pM to about 10 pM, or about 10 pM to about 1 pM.

According to a particular aspect, the invention relates to an isolated monoclonal antibody or antigen-binding fragment thereof, wherein the monoclonal antibody or antigen-binding fragment thereof or antigen binding domain comprises a heavy chain complementarity determining region 1 (HCDR1), a HCDR2, a HCDR3, a light chain complementarity determining region 1 (LCDR1), a LCDR2, and a LCDR3, having the polypeptide sequences of:
(1) SEQ ID NOs: 1, 2, 3, 4, 5, and 6, respectively;
(2) SEQ ID NOs: 13, 2, 14, 4, 28, and 6, respectively;
(3) SEQ ID NOs: 13, 2, 15, 4, 5, and 6, respectively;
(4) SEQ ID NOs: 16, 17, 18, 29, 30, and 6, respectively;
(5) SEQ ID NOs: 19, 20, 21, 4, 5, and 6, respectively;
(6) SEQ ID NOs: 22, 23, 24, 31, 5, and 32, respectively; or
(7) SEQ ID NOs: 25, 26, 27, 33, 34, and 35, respectively;
   wherein the antibody or antigen-binding fragment thereof or antigen binding domain thereof specifically binds chemokine (C—C motif) receptor 8 (CCR8), preferably human CCR8.

According to another particular aspect, the invention relates to an isolated monoclonal antibody or antigen-binding fragment thereof, wherein the monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:7, 36, 38, 40, 42, 44, or 46, or a light chain variable region having a polypeptide sequence at least 85%, preferably 90%, more preferably 95% or more, such as 95%, 96%, 97%, 98%, or 99% identical to SEQ ID NO:8, 37, 39, 41, 43, 45, or 47. According to one preferred embodiment, the isolated monoclonal antibody or antigen-binding fragment thereof of the invention comprises a heavy chain variable region having the polypeptide sequence at least 85%, preferably 90%, more preferably 95% or more, such as 95%, 96%, 97%, 98%, or 99% identical to SEQ ID NO: 7, and a light chain variable region having a polypeptide sequence at least 85%, preferably 90%, more preferably 95% or more, such as 95%, 96%, 97%, 98%, or 99% identical to SEQ ID NO: 8, respectively.

According to a particular embodiment, the isolated anti-CCR8 monoclonal antibody or antigen-binding fragment thereof comprises:
(1) a heavy chain variable region having the polypeptide sequence of SEQ ID NO:7, and a light chain variable region having the polypeptide sequence of SEQ ID NO:8;
(2) a heavy chain variable region having the polypeptide sequence of SEQ ID NO:36, and a light chain variable region having the polypeptide sequence of SEQ ID NO:37;
(3) a heavy chain variable region having the polypeptide sequence of SEQ ID NO:38, and a light chain variable region having the polypeptide sequence of SEQ ID NO:39;
(4) a heavy chain variable region having the polypeptide sequence of SEQ ID NO:40, and a light chain variable region having the polypeptide sequence of SEQ ID NO:41;
(5) a heavy chain variable region having the polypeptide sequence of SEQ ID NO:42, and a light chain variable region having the polypeptide sequence of SEQ ID NO:43;
(6) a heavy chain variable region having the polypeptide sequence of SEQ ID NO:44, and a light chain variable region having the polypeptide sequence of SEQ ID NO:45; or
(7) a heavy chain variable region having the polypeptide sequence of SEQ ID NO:46, and a light chain variable region having the polypeptide sequence of SEQ ID NO:47.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 1, 2, 3, 4, 5, and 6, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:7, and a light chain variable region having a polypeptide sequence at least 85%, preferably 90%, more preferably 95% or more, such as 95%, 96%, 97%, 98%, or 99% identical to SEQ ID NO:8. Preferably, the isolated monoclonal antibody or antigen-binding fragment thereof comprises a heavy chain variable region having the polypeptide sequence of SEQ ID NO:7; and a light chain variable region having the polypeptide sequence of SEQ ID NO:8.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 13, 2, 14, 4, 28, and 6, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:36, and a light chain variable region having a polypeptide sequence at least 85%, preferably 90%, more preferably 95% or more, such as 95%, 96%, 97%, 98%, or 99% identical to SEQ ID NO:37. Preferably, the isolated monoclonal antibody or antigen-binding fragment thereof comprises a heavy chain variable region having the polypeptide sequence of SEQ ID NO:36; and a light chain variable region having the polypeptide sequence of SEQ ID NO:37.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 13, 2, 15, 4, 5, and 6, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:38, and a light chain variable region having a polypeptide sequence at least 85%, preferably 90%, more preferably 95% or more, such as 95%, 96%, 97%, 98%, or 99% identical to SEQ ID NO:39. Preferably, the isolated monoclonal antibody or antigen-binding fragment thereof comprises a heavy chain variable region having the polypeptide sequence of SEQ ID NO:38; and a light chain variable region having the polypeptide sequence of SEQ ID NO:39.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 16, 17, 18, 29, 30, and 6, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 40, and a light chain variable region having a polypeptide sequence at least 85%, preferably 90%, more preferably 95% or more, such as 95%, 96%, 97%, 98%, or 99% identical to SEQ ID NO: 41. Preferably, the isolated monoclonal antibody or antigen-binding fragment thereof comprises a heavy chain variable region having the polypeptide sequence of SEQ ID NO:40; and a light chain variable region having the polypeptide sequence of SEQ ID NO:41.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 19, 20, 21, 4, 5, and 6, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:42, and a light chain variable region having a polypeptide sequence at least 85%, preferably 90%, more preferably 95% or more, such as 95%, 96%, 97%, 98%, or 99% identical to SEQ ID NO:43. Preferably, the isolated monoclonal antibody or antigen-binding fragment thereof comprises a heavy chain variable region having the polypeptide sequence of SEQ ID NO:42; and a light chain variable region having the polypeptide sequence of SEQ ID NO:43.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 22, 23, 24, 31, 5, and 32, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 44, and a light chain variable region having a polypeptide sequence at least 85%, preferably 90%, more preferably 95% or more, such as 95%, 96%, 97%, 98%, or 99% identical to SEQ ID NO: 45. Preferably, the isolated monoclonal antibody or antigen-binding fragment thereof comprises a heavy chain variable region having the polypeptide sequence of SEQ ID NO:44; and a light chain variable region having the polypeptide sequence of SEQ ID NO:45.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 25, 26, 27, 33, 34, and 35, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 46, and a light chain variable region having a polypeptide sequence at least 85%, preferably 90%, more preferably 95% or more, such as 95%, 96%, 97%, 98%, or 99% identical to SEQ ID NO: 47. Preferably, the isolated monoclonal antibody or antigen-binding fragment thereof comprises a heavy chain variable region having the polypeptide sequence of SEQ ID NO:46; and a light chain variable region having the polypeptide sequence of SEQ ID NO:47.

According to another particular aspect, the invention relates to an isolated monoclonal antibody or antigen-binding fragment thereof or a bispecific antibody or antigen-binding fragment thereof of the invention, wherein the monoclonal or bispecific antibody or antigen-binding fragment thereof is chimeric.

According to another particular aspect, the invention relates to an isolated monoclonal antibody or antigen-binding fragment thereof or a bispecific antibody or antigen-binding fragment thereof of the invention, wherein the monoclonal or bispecific antibody or antigen-binding fragment thereof is human or humanized.

According to another particular aspect, the invention relates to a humanized monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 1, 48, 49, 50, 51, and 6, respectively;
(2) SEQ ID NOs: 1, 2, 49, 4, 5, and 6, respectively;
(3) SEQ ID NOs: 1, 2, 49, 50, 51, and 6, respectively;
(4) SEQ ID NOs: 1, 2, 3, 4, 5, and 6, respectively; or
(5) SEQ ID NOs: 1, 48, 49, 50, 51, and 6, respectively.

According to another particular aspect, the invention relates to a humanized monoclonal antibody or antigen-binding fragment thereof, wherein the monoclonal antibody or antigen-binding fragment thereof comprises a heavy chain variable region having a polypeptide sequence at least 90%, at least 95%, at least 96%, at least 97%, at least 98%, or at least 99% identical to SEQ ID NO:9, 52, 53, 54, 55, 56, 57, 58, 59, or 60, or a light chain variable region having a polypeptide sequence at least 90%, at least 95%, at least 96%, at least 97%, at least 98%, or at least 99% identical to SEQ ID NO:10, 61, 62, or 63.

According to another particular aspect, the humanized anti-CCR8 monoclonal antibody or antigen-binding fragment thereof comprises:
(1) a heavy chain variable region having the polypeptide sequence of SEQ ID NO:9, and a light chain variable region having the polypeptide sequence of SEQ ID NO:10;
(2) a heavy chain variable region having the polypeptide sequence of SEQ ID NO:52, and a light chain variable region having the polypeptide sequence of SEQ ID NO:63;
(3) a heavy chain variable region having the polypeptide sequence of SEQ ID NO:52, and a light chain variable region having the polypeptide sequence of SEQ ID NO:10;
(4) a heavy chain variable region having the polypeptide sequence of SEQ ID NO:53, and a light chain variable region having the polypeptide sequence of SEQ ID NO:10;
(5) a heavy chain variable region having the polypeptide sequence of SEQ ID NO:54, and a light chain variable region having the polypeptide sequence of SEQ ID NO:63; or
(6) a heavy chain variable region having the polypeptide sequence of SEQ ID NO:59, and a light chain variable region having the polypeptide sequence of SEQ ID NO:62.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 1, 48, 49, 50, 51, and 6, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:9, and a light chain variable region having a polypeptide sequence at least 85%, preferably 90%, more preferably 95% or more, such as 95%, 96%, 97%, 98%, or 99% identical to SEQ ID NO:10. Preferably, the isolated monoclonal antibody or antigen-binding fragment thereof comprises a heavy chain variable region having the polypeptide sequence of SEQ ID NO:9; and a light chain variable region having the polypeptide sequence of SEQ ID NO:10.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 1, 2, 49, 4, 5, and 6, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:52, and a light chain variable region having a polypeptide sequence at least 85%, preferably 90%, more preferably 95% or more, such as 95%, 96%, 97%, 98%, or 99% identical to SEQ ID NO:63. Preferably, the isolated monoclonal antibody or antigen-binding fragment thereof comprises a heavy chain variable region having the polypeptide sequence of SEQ ID NO:52; and a light chain variable region having the polypeptide sequence of SEQ ID NO:63.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 1, 2, 49, 50, 51, and 6, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:52, and a light chain variable region having a polypeptide sequence at least 85%, preferably 90%, more preferably 95% or more, such as 95%, 96%, 97%, 98%, or 99% identical to SEQ ID NO:10. Preferably, the isolated monoclonal antibody or antigen-binding fragment thereof comprises a heavy chain variable region having the polypeptide sequence of SEQ ID NO:52; and a light chain variable region having the polypeptide sequence of SEQ ID NO: 10.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 1, 2, 49, 50, 51, and 6, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:53, and a light chain variable region having a polypeptide sequence at least 85%, preferably 90%, more preferably 95% or more, such as 95%, 96%, 97%, 98%, or 99% identical to SEQ ID NO:10. Preferably, the isolated monoclonal antibody or antigen-binding fragment thereof comprises a heavy chain variable region having the polypeptide sequence of SEQ ID NO:53; and a light chain variable region having the polypeptide sequence of SEQ ID NO: 10.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 1, 2, 3, 4, 5, and 6, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:54, and a light chain variable region having a polypeptide sequence at least 85%, preferably 90%, more preferably 95% or more, such as 95%, 96%, 97%, 98%, or 99% identical to SEQ ID NO:63.

Preferably, the isolated monoclonal antibody or antigen-binding fragment thereof comprises a heavy chain variable region having the polypeptide sequence of SEQ ID NO:54; and a light chain variable region having the polypeptide sequence of SEQ ID NO:63.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 1, 48, 49, 50, 51, and 6, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:59, and a light chain variable region having a polypeptide sequence at least 85%, preferably 90%, more preferably 95% or more, such as 95%, 96%, 97%, 98%, or 99% identical to SEQ ID NO:62. Preferably, the isolated monoclonal antibody or antigen-binding fragment thereof comprises a heavy chain variable region having the polypeptide sequence of SEQ ID NO:59; and a light chain variable region having the polypeptide sequence of SEQ ID NO:62.

In another general aspect, the invention relates to an isolated nucleic acid encoding a monoclonal antibody or antigen-binding fragment thereof and/or a bispecific antibody or antigen-binding fragment thereof of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding monoclonal antibodies or antigen-binding fragments thereof of the invention can be altered without changing the amino acid sequences of the proteins.

In another general aspect, the invention relates to a vector comprising an isolated nucleic acid encoding a monoclonal antibody or antigen-binding fragment thereof and/or a bispecific antibody or antigen-binding fragment thereof of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention.

In another general aspect, the invention relates to a host cell comprising an isolated nucleic acid encoding a monoclonal antibody or antigen-binding fragment thereof and/or a bispecific antibody or antigen-binding fragment thereof of the invention. Any host cell known to those skilled in the art in view of the present disclosure can be used for recombinant expression of antibodies or antigen-binding fragments thereof of the invention. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, the invention relates to a method of producing a monoclonal antibody or antigen-binding fragment thereof and/or a bispecific antibody or antigen-binding fragment thereof of the invention, comprising culturing a cell comprising a nucleic acid encoding the monoclonal antibody or antigen-binding fragment thereof or bispecific antibody or antigen-binding fragment thereof under conditions to produce a monoclonal antibody or antigen-binding fragment thereof or bispecific antibody or antigen-binding fragment thereof of the invention, and recovering the monoclonal and/or bispecific antibody or antigen-binding fragment thereof from the cell or cell culture (e.g., from the supernatant). Expressed monoclonal and/or bispecific antibodies or antigen-binding fragments thereof can be harvested from the cells and purified according to conventional techniques known in the art and as described herein.

### Pharmaceutical Compositions

In another general aspect, the invention relates to a pharmaceutical composition, comprising an isolated monoclonal antibody or antigen-binding fragment thereof, a bispecific antibody or antigen-binding fragment thereof, an isolated polynucleotide, and/or an isolated polypeptide of the invention and a pharmaceutically acceptable carrier.

The term “pharmaceutical composition” as used herein means a product comprising an isolated polynucleotide of the invention, an isolated polypeptide of the invention, an anti-CCR8 monoclonal antibody or antigen-binding fragment thereof, and/or a bispecific antibody of the invention together with a pharmaceutically acceptable carrier. Polynucleotides, polypeptides, an anti-CCR8 monoclonal antibody or antigen-binding fragment thereof, and/or a bispecific antibody of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in an antibody pharmaceutical composition can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include buffers, diluents, solvents, tonicity regulating agents, preservatives, stabilizers, and chelating agents. One or more pharmaceutically acceptable carrier(s) can be used in formulating the pharmaceutical compositions of the invention.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms may be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment of the invention, the pH of the formulation is from about 7.0 to about 9.5. In another embodiment of the invention, the pH of the formulation is from about 3.0 to about 7.0.

In another embodiment of the invention, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments of the invention.

In another embodiment of the invention, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments of the invention.

In another embodiment of the invention, the pharmaceutical composition comprises an isotonic agent. Non-limiting examples of isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g., PEG400), and mixtures thereof. Another example of an isotonic agent includes a sugar. Non-limiting examples of sugars may be mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethylcellulose. Another example of an isotonic agent is a sugar alcohol, wherein the term “sugar alcohol” is defined as a C(4-8) hydrocarbon having at least one-OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments of the invention.

In another embodiment of the invention, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments of the invention.

In another embodiment of the invention, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment of the invention, the pharmaceutical composition comprises a stabilizer, wherein said stabilizer is carboxy-/hydroxycellulose and derivatives thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments of the invention.

In further embodiments of the invention, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments of the invention.

In a further embodiment of the invention, the pharmaceutical composition comprises one or more protease inhibitors, such as, e.g., EDTA, and/or benzamidine hydrochloric acid (HCl). The protease inhibitor can be present individually or in the aggregate, in a concentration from about 0.1 mg/ml to about 20 mg/ml. Pharmaceutical compositions comprising each one of these specific protease inhibitors constitute alternative embodiments of the invention.

In another general aspect, the invention relates to a method of producing a pharmaceutical composition comprising a monoclonal antibody or antigen-binding fragment thereof and/or a bispecific antibody or antigen-binding fragment thereof of the invention, comprising combining a monoclonal antibody or antigen-binding fragment thereof and/or a bispecific antibody or antigen-binding fragment thereof with a pharmaceutically acceptable carrier to obtain the pharmaceutical composition.

### Methods of Use

In another general aspect, the invention relates to a method of treating a cancer in a subject in need thereof, comprising administering to the subject the pharmaceutical compositions comprising anti-CCR8 monoclonal and/or bispecific antibodies or antigen binding fragments thereof of the invention. The cancer can, for example, be selected from, but not limited to, lung cancer, head and neck cancer, esophageal cancer, stomach cancer, colorectal cancer, breast cancer, pancreatic cancer, ovarian cancer, kidney cancer, and melanoma.

In some aspects, the invention related to a method of inducing tumor infiltrating regulatory T cell (TITC) depletion in a subject in need thereof, comprising administering to the subject the pharmaceutical compositions comprising anti-CCR8 monoclonal and/or bispecific antibodies or antigen binding fragments thereof of the invention. In various aspects, the other T cells, including CD4+ T cells and CD8+ T cells are not affected by the induced cell depletion. In other aspects, the method further induces a reprogramming of tumor microenvironment. In various aspects, inducing TITC depletion includes inducing natural killer (NK)-mediated killing of CCR-8 expressing cancer cells. In other aspects, inducing NK-mediated killing includes inhibiting TITC-induced immunosuppression.

In another general aspect, the invention relates to a method of targeting CCR8 on a cancer cell surface in a subject to achieve cell killing, the method comprising administering to the subject an isolated monoclonal antibody or antigen binding fragment thereof and/or bispecific antibody or antigen-binding fragment thereof that specifically binds CCR8 or a pharmaceutical composition comprising the isolated monoclonal antibody or antigen binding fragment thereof and/or bispecific antibody or antigen-binding fragment thereof of the invention. Binding of the anti-CCR8 monoclonal or bispecific antibody or antigen-binding fragment to CCR8 can mediate antibody-dependent cellular phagocytosis (ADCP), and/or antibody-dependent cellular cytotoxicity (ADCC) or other effects that result in the death of the targeted cancer cell. The monoclonal or bispecific antibody or antigen binding fragment thereof can, for example, serve to recruit conjugated drugs, and/or can form a bispecific antibody with another monoclonal antibody to mediate the death of the targeted cancer cell.

The functional activity of antibodies and antigen-binding fragments thereof that bind CCR8 can be characterized by methods known in the art and as described herein. Methods for characterizing antibodies and antigen-binding fragments thereof that bind CCR8 include, but are not limited to, affinity and specificity assays including Biacore, ELISA, and OctetRed analysis, and detection of the binding of antibodies and antigen-binding fragments to CCR8 on cells (either cells transfected with CCR8 or cells that naturally express CCR8) by FACS. According to particular embodiments, the methods for characterizing antibodies and antigen-binding fragments thereof that bind CCR8 include those described below.

In another general aspect, the invention relates to a method of treating a cancer in a subject in need thereof, comprising administering to the subject an isolated monoclonal antibody or antigen-binding fragment thereof and/or bispecific antibody or antigen-binding fragment thereof that specifically binds CCR8 or a pharmaceutical composition of the invention. The cancer can, for example, be a solid tumor, preferably a solid tumor with infiltrating T cells, more preferably a solid tumor with infiltrating T reg cells, more preferably a solid tumor with highly suppressive T reg cells expressing CCR8, most preferably a solid tumor with infiltrating highly suppressive T reg cells overexpressing CCR8 for which natural killer (NK) cell infiltration has occurred. Cancers can, for example, be selected from, but not limited to, lung cancer, head and neck cancer, esophageal cancer, stomach cancer, colorectal cancer, breast cancer, pancreatic cancer, ovarian cancer, bladder cancer, liver cancer, kidney cancer, and melanoma. According to particular embodiments, the subject can, for example, comprise CCR8-expressing Tregs.

As used herein with reference to anti-CCR8 antibodies or antigen-binding fragments thereof, a therapeutically effective amount means an amount of the anti-CCR8 antibody or antigen-binding fragment thereof that modulates an immune response in a subject in need thereof. Also, as used herein with reference to anti-CCR8 antibodies or antigen-binding fragments thereof, a therapeutically effective amount means an amount of the anti-CCR8 antibody or antigen-binding fragment thereof that results in treatment of a disease, disorder, or condition; prevents or slows the progression of the disease, disorder, or condition; or reduces or completely alleviates symptoms associated with the disease, disorder, or condition.

According to particular embodiments, the disease, disorder or condition to be treated is cancer. The cancer can, for example, be a solid tumor, preferably a solid tumor with infiltrating T cells, more preferably a solid tumor with infiltrating T reg cells, more preferably a solid tumor with highly suppressive T reg cells expressing CCR8, most preferably a solid tumor with infiltrating highly suppressive T reg cells overexpressing CCR8 for which natural killer (NK) cell infiltration has occurred. The cancer can, for example, be selected from, but not limited to, lung cancer, head and neck cancer, esophageal cancer, stomach cancer, colorectal cancer, breast cancer, pancreatic cancer, ovarian cancer, bladder cancer, liver cancer, kidney cancer, and melanoma.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment of a cancer. For cancer therapy, the provided compositions can be used in combination with another treatment including, but not limited to, a chemotherapy, an anti-CD20 mAb, an anti-EGFR mAb, an anti-HER-2 mAb, an anti-CD19 mAb, an anti-CD33 mAb, an anti-CD47 mAb, an anti-CD73 mAb, an anti-PD-1 mAb, an anti-PD-L1 mAb, an anti-CTLA mAb, an anti-TNFR2 mAb, an anti-OX40 mAb, other immuno-oncology drugs, an antiangiogenic agent, a radiation therapy, an antibody-drug conjugate (ADC), a targeted therapy, other anticancer drugs, and/or treatments targeting immunomodulatory targets including, but not limited to PD1 and PD-L1. Antibodies against CCR8 can be used to construct bispecific antibodies with partner mAbs against PD-1, PD-L1, CTLA-4, CTLA, TNFR2, OX40, EGFR, HER-2, CD19, CD20, CD33, CD73, CD47, and/or CD3. Two antibodies that recognize two different epitopes on CCR8 can also be used to construct a bispecific antibody to treat cancers/tumors that express CCR8.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

In another general aspect, the invention relates to a method of determining a level of a CCR8 in a subject. The methods comprise (a) obtaining a sample from the subject; (b) contacting the sample with a monoclonal antibody or antigen-binding fragment thereof of the invention; and (c) determining a level of CCR8 in the subject.

As used herein, “sample” refers to a biological sample isolated from a subject and can include, but is not limited to, whole blood, serum, plasma, blood cells, endothelial cells, tissue biopsies (e.g., a cancer tissue), lymphatic fluid, ascites fluid, interstitial fluid, bone marrow, cerebrospinal fluid, saliva, mucous, sputum, sweat, urine, or any other secretion, excretion, or other bodily fluids. A “blood sample” refers to whole blood or any fraction thereof, including blood cells, serum, and plasma. A sample can, for example, comprise Treg cells.

In certain embodiments, the level of CCR8 in the subject can be determined utilizing assays selected from, but not limited to, a Western blot assay, immunohistochemistry (IHC) and an ELISA. Relative protein levels can be determined by utilizing Western blot analysis and IHC, and absolute protein levels can be determined by utilizing an ELISA. When determining the relative levels of CCR8, the levels of CCR8 can be determined between at least two samples, e.g., between samples from the same subject at different time points, between samples from different tissues in the same subject, and/or between samples from different subjects. Alternatively, when determining absolute levels of CCR8, such as by an ELISA, the absolute level of CCR8 in the sample can be determined by creating a standard for the ELISA prior to testing the sample. A person skilled in the art would understand which analytical techniques to utilize to determine the level of CCR8 in a sample from the subject utilizing the antibodies or antigen-binding fragments thereof of the invention.

Utilizing methods of determining a level of CCR8 in a sample from a subject can lead to the diagnosis of abnormal (elevated, reduced, or insufficient) CCR8 levels in a disease and making appropriate therapeutic decisions. Such a disease can be a cancer. Additionally, by monitoring the levels of CCR8 in a subject, the risk of developing a disease as indicated above can be determined based on the knowledge of the level of CCR8 in a particular disease and/or during the progression of the particular disease.

### EMBODIMENTS

The invention provides also the following non-limiting embodiments.

Embodiment 1 is an isolated monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 1, 2, 3, 4, 5, and 6, respectively;
(2) SEQ ID NOs: 13, 2, 14, 4, 28, and 6, respectively;
(3) SEQ ID NOs: 13, 2, 15, 4, 5, and 6, respectively;
(4) SEQ ID NOs: 16, 17, 18, 29, 30, and 6, respectively;
(5) SEQ ID NOs: 19, 20, 21, 4, 5, and 6, respectively;
(6) SEQ ID NOs: 22, 23, 24, 31, 5, and 32, respectively; or
(7) SEQ ID NOs: 25, 26, 27, 33, 34, and 35, respectively;
   wherein the antibody or antigen-binding fragment thereof specifically binds CCR8, preferably specifically binds human CCR8.

Embodiment 2 is the isolated monoclonal antibody or antigen-binding fragment thereof of embodiment 1, comprising a heavy chain variable region having a polypeptide sequence at least 95% identical to SEQ ID NO: 7, 36, 38, 40, 42, 44, or 46, or a light chain variable region having a polypeptide sequence at least 95% identical to SEQ ID NO: 8, 37, 39, 41, 43, 45, or 47.

Embodiment 3 is the isolated monoclonal antibody or antigen-binding fragment thereof of embodiment 1 or 2, comprising:
(1) a heavy chain variable region having the polypeptide sequence of SEQ ID NO:7, and a light chain variable region having the polypeptide sequence of SEQ ID NO:8;
(2) a heavy chain variable region having the polypeptide sequence of SEQ ID NO:36, and a light chain variable region having the polypeptide sequence of SEQ ID NO:37;
(3) a heavy chain variable region having the polypeptide sequence of SEQ ID NO:38, and a light chain variable region having the polypeptide sequence of SEQ ID NO:39;
(4) a heavy chain variable region having the polypeptide sequence of SEQ ID NO:40, and a light chain variable region having the polypeptide sequence of SEQ ID NO:41;
(5) a heavy chain variable region having the polypeptide sequence of SEQ ID NO:42, and a light chain variable region having the polypeptide sequence of SEQ ID NO:43;
(6) a heavy chain variable region having the polypeptide sequence of SEQ ID NO:44, and a light chain variable region having the polypeptide sequence of SEQ ID NO:45; or
(7) a heavy chain variable region having the polypeptide sequence of SEQ ID NO:46, and a light chain variable region having the polypeptide sequence of SEQ ID NO:47.

Embodiment 4 is the isolated monoclonal antibody or antigen-binding fragment thereof of any one of embodiments 1-3, wherein the antibody or antigen-binding fragment thereof is chimeric and/or human or humanized.

Embodiment 5 is the isolated monoclonal antibody or antigen-binding fragment thereof of embodiment 4, wherein the isolated monoclonal antibody or antigen-binding fragment thereof comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 1, 48, 49, 50, 51, and 6, respectively;
(2) SEQ ID NOs: 1, 2, 49, 4, 5, and 6, respectively;
(3) SEQ ID NOs: 1, 2, 49, 50, 51, and 6, respectively;
(4) SEQ ID NOs: 1, 2, 3, 4, 5, and 6, respectively; or
(5) SEQ ID NOs: 1, 48, 49, 50, 51, and 6.

Embodiment 6 is the isolated monoclonal antibody or antigen-binding fragment thereof of embodiment 5, wherein the isolated monoclonal antibody or antigen-binding fragment thereof comprises a heavy chain variable region having a polypeptide sequence at least 95% identical to SEQ ID NO:9, 52, 53, 54, 55, 56, 57, 58, 59, or 60, or a light chain variable region having a polypeptide sequence at least 95% identical to SEQ ID NO:10, 61, 62, or 63.

Embodiment 7 is the isolated monoclonal antibody or antigen-binding fragment thereof of embodiment 6, wherein the isolated monoclonal antibody or antigen-binding fragment thereof comprises:
(1) a heavy chain variable region having the polypeptide sequence of SEQ ID NO:9, and a light chain variable region having the polypeptide sequence of SEQ ID NO:10;
(2) a heavy chain variable region having the polypeptide sequence of SEQ ID NO:52, and a light chain variable region having the polypeptide sequence of SEQ ID NO:63;
(3) a heavy chain variable region having the polypeptide sequence of SEQ ID NO:52, and a light chain variable region having the polypeptide sequence of SEQ ID NO:10;
(4) a heavy chain variable region having the polypeptide sequence of SEQ ID NO:53, and a light chain variable region having the polypeptide sequence of SEQ ID NO:10;
(5) a heavy chain variable region having the polypeptide sequence of SEQ ID NO:54, and a light chain variable region having the polypeptide sequence of SEQ ID NO:63; or
(6) a heavy chain variable region having the polypeptide sequence of SEQ ID NO:59, and a light chain variable region having the polypeptide sequence of SEQ ID NO:62.

Embodiment 8 is the isolated monoclonal antibody or antigen-binding fragment thereof of any one of embodiments 1-7, wherein the isolated antibody or antigen-binding fragment thereof is capable of inducing effector-mediated tumor cell lysis through antibody-dependent cellular cytotoxicity (ADCC); antibody-dependent cellular phagocytosis (ADCP); and/or mediating the recruitment of conjugated drugs; and/or forming a bispecific antibody with another mAb or antigen-binding fragment thereof with cancer-killing effect.

Embodiment 9 is the isolated monoclonal antibody or antigen-binding fragment thereof of any one of embodiments 1-8, wherein the monoclonal antibody or antigen-binding fragment thereof specifically binds cynomolgus CCR8.

Embodiment 10 is an isolated bispecific antibody or antigen-binding fragment thereof comprising the monoclonal antibody or antigen-binding fragment thereof of any one of embodiments 1-9.

Embodiment 11 is an isolated nucleic acid encoding the monoclonal antibody or antigen-binding fragment of any one of embodiments 1-9.

Embodiment 12 is an isolated nucleic acid encoding the bispecific antibody or antigen-binding fragment thereof of embodiment 10.

Embodiment 13 is a vector comprising the isolated nucleic acid of embodiment 11 or 12.

Embodiment 14 is a host cell comprising the vector of embodiment 13.

Embodiment 15 is a pharmaceutical composition, comprising the isolated monoclonal antibody or antigen-binding fragment of any one of embodiments 1-9 or the bispecific antibody or antigen-binding fragment thereof of embodiment 10 and a pharmaceutically acceptable carrier.

Embodiment 16 is a method of targeting CCR8 on a cancer cell surface, and/or treating a cancer, comprising administering to the subject the pharmaceutical composition of embodiment 15.

Embodiment 17 is the method of embodiment 16, wherein the cancer is a solid tumor, preferably a solid tumor with infiltrating T cells, more preferably a solid tumor with infiltrating T reg cells, more preferably a solid tumor with highly suppressive T reg cells expressing CCR8, most preferably a solid tumor with infiltrating highly suppressive T reg cells overexpressing CCR8 for which natural killer (NK) cell infiltration has occurred.

Embodiment 18 is the method of embodiment 16 or 17, wherein the cancer is selected from the group consisting of lung cancer, head and neck cancer, esophageal cancer, stomach cancer, colorectal cancer, breast cancer, pancreatic cancer, ovarian cancer, kidney cancer, and melanoma.

Embodiment 19 is the method of any one of embodiments 16 to 18, wherein the subject comprises CCR8-expressing Treg cells.

Embodiment 20 is a method of producing the monoclonal antibody or antigen-binding fragment of any one of embodiments 1-9 or the bispecific antibody or antigen-binding fragment thereof of embodiment 10, comprising culturing a cell comprising a nucleic acid encoding the monoclonal antibody or antigen-binding fragment thereof or bispecific antibody or antigen-binding fragment thereof under conditions to produce the monoclonal antibody or antigen-binding fragment thereof or bispecific antibody or antigen-binding fragment thereof and recovering the monoclonal antibody or antigen-binding fragment thereof or bispecific antibody or antigen-binding fragment thereof from the cell or culture.

Embodiment 21 is a method of producing a pharmaceutical composition comprising the monoclonal antibody or antigen-binding fragment thereof of any one of embodiments 1-9 or the bispecific antibody or antigen-binding fragment thereof of embodiment 10, comprising combining the monoclonal antibody or antigen-binding fragment thereof or bispecific antibody or antigen-binding fragment thereof with a pharmaceutically acceptable carrier to obtain the pharmaceutical composition.

Embodiment 22 is a method of determining the level of CCR8 in a subject, the method comprising:
a. obtaining a sample from the subject;
b. contacting the sample with the isolated monoclonal antibody or antigen-binding fragment thereof of any one of embodiments 1-9; and
c. determining the level of the CCR8 in the subject.

Embodiment 23 is the method of embodiment 22, wherein the sample is a tissue sample or a blood sample, optionally wherein the tissue sample is a cancer tissue sample.

Embodiment 24 is the method of embodiment 22, wherein the sample comprises Treg cells.

### EXAMPLES

### Example 1: Antibody Preparation

### Immunization:

Anti-CCR antibodies were developed by immunizing Balb/c mice with four DNA injections followed by two to four boosts of stable M300.19 cells expressing hCCR8 receptor. Mice showing high specific titer (>10,000) were processed into single B-cell sorting in CelliGo™ platform.

### B-Cell Sorting by CelliGo Platform:

Splenocytes from immunized mice were activated in vitro in X-vivo 15 media (supplemented by 1% Penicillin/Streptomycin, 2× L-glutamine, 10% low IgG sera, 100 ng/ml, IL-2 and 2.5 μg/mL R848) for 5 days at 37° C., 5% CO2. B-cells were enriched using Pan B isolation kit (Miltenyi Biotec; Bergisch Gladback, North-Rhine-Westphalia, Germany) and labeled with Celltrace™ Violet reagent (Thermofisher; Waltham, MA) before sorting using HiFiBio Celligo platform (HiFiBio; Cambridge, MA). A cell-based screening assay was developed using HiFiBio microfluidic technology to detect and sort specific B-cells that bind to the target cell in the droplet.

VH/VL were amplified from single B-cells in droplet by RT-PCR and PCR, and the sequences were analyzed using Absolution HiFiBio software (HiFiBio).

Chimeric antibodies were produced in human IgG1 scaffold and screened by fluorescence-activated cell sorting (FACS) for specific binding on CCR8 high copy cells (stable CCR8 CHO-K1 cell line, Perkin Elmer; Waltham, MA), compared to parental cells.

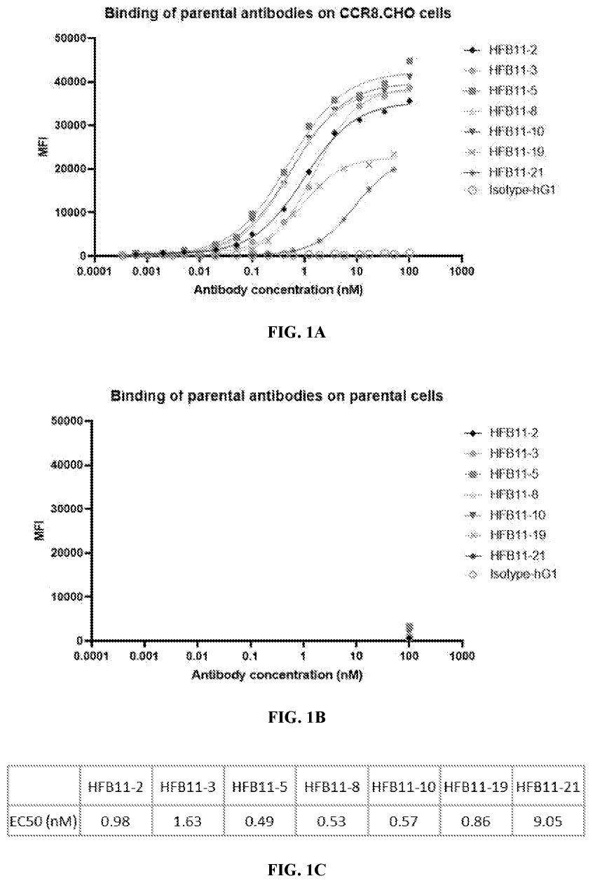

Among 25 specific anti-hCCR8 antibodies, a panel of antibodies with variable biological properties was identified (FIGS. 1-6).

The sequences for the antibodies are provided below.

TABLE 1
Sequences of heavy chain variable regions for anti-CCR8 mAbs.

| | | SEQ |
|---|---|---|
| mAb | | ID |
| clones | VH sequence | NO: |
| HFB11-10 | EVQLVETGGGLVQPKGSLKLSCAASGFRFNTNAMNWVRQAPGKDLEWVARIRSKS | 7 |
| | NNYATYYADSVKDRFTISRDDSQSMLYLQMNNVKTEDTAMYYCVRGSDNNYYYA | |
| | MDYWGQGTSVTVSS | |
| HFB11-2 | EVQLVESGGGLVQPKGSLKLSCAASGFSFNTYAMNWVRQAPGKGLEWVARIRSKSN | 36 |
| | NYATYYADSVKDRFTISRDDSERMLYLQMNNLKTEDTAMYYCVRGGSYYSNHYFD | |
| | YWGQGTTLTVSS | |
| HFB11-3 | EVQLVESGGGLVQPQGSLKLSCAASGFSFNTYAMNWVRQAPGKGLQWVARIRSKS | 38 |
| | NNYATYYADSVKDRFTISRDDSESMLYLQMNNLKTEDAAMYYCVRGKDANYFYA | |
| | MDYWGQGTSVTVSS | |
| HFB11-5 | EVQLVESGGGLVQPRGSLKLSCTASGFSFNAYAMNWVRQAPGKGLEWVARIRTKSN | 40 |
| | NYATYYADSVKDRFTISRDDSESMLYLQMKNLKTEDTAMYYCVRGGYGNGGYYFD | |
| | YWGQGTTLTVSS | |
| HFB11-8 | EVQLVETGGGLVQPKGSLKLSCAASGFRFNTNAMNWVRQAPGKGLEWVARIRSKS | 42 |
| | NNYATHYVDSVKDRFTVSRDDSQSMLYLQMNNLKAEDTAMYYCVRGSDNNYYYA | |
| | MDYWGQGTSVTVSS | |
| HFB11-19 | QVQLQQSGAELVKPGASVKLSCKASGYTFTDYIIHWVKQRSGQGLEWIGWFSPRNN | 44 |
| | YVKYNERFTDKATLTADKSSNTVYMELSSLTSEDSAVYFCSRHGRNSIGFAYWGQG | |
| | TLVTVSA | |
| HFB11-21 | QVQLQQPGAELVKPGASVKLSCKASGYTFISYWMHWVKQRPGRGLEWIGRIDPNSG | 46 |
| | KTKYNEKFKTKATLTVDKSSSTAYMHLNSLTSDDSAVYYCAREGWGDYWGQGTTL | |
| | TVSS | |

TABLE 2
Sequences of light chain variable regions for anti-CCR8 mAbs.

| | | SEQ |
|---|---|---|
| mAb | | ID |
| clones | VL sequence | NO: |
| HFB11-10 | DIVMTQAAPSVPVTPGESVSISCRSSKSLLHSNGNTYLYWFLQRPGQSPQLLIYRMS | 8 |
| | NLASGVPDRFSGSGSGTAFTLRISRVEAEDVGVYYCMQHLEYPFTFGSGTKLEIK | |
| HFB11-2 | DIVMTQAAPSVPVTPGESVSISCRSSKSLLHSNGNTYLYWFLQRPGQSPQLLIYQMS | 37 |
| | NLASGVPDRFSGSGSGTAFTLRISRVEAEDVGVYYCMQHLEYPFTFGAGTKLELK | |
| HFB11-3 | DIVMTQAAPSVPVTPGESVSISCRSSKSLLHSNGNTYLYWFLQRPGQSPQLLIYRMS | 39 |
| | NLASGVPDRFSGSGSGTAFTLRISRVEAEDVGVYYCMQHLEYPFTFGAGTKLELK | |
| HFB11-5 | DIVMTQAAPSVTVTPGESASISCRSSKSLLHRNGNSYLYWFLQRPGQSPQLLIYRIS | 41 |
| | NLASGVPDRFSGSGSGTAFTLRISRVEAEDVGVYYCMQHLEYPFTFGSGTKLEIK | |
| HFB11-8 | DIVMTQAAPSVPVTPGESVSISCRSSKSLLHSNGNTYLYWFLQRPGQSPQLLIYRMS | 43 |
| | NLASGVPDRFSGSGSGTSFTLRISRVEVEDVGVYYCMQHLEYPFTFGSGTKLEIK | |
| HFB11-19 | DIVMTQAAPSVPVTPGESVSISCRSSKSLLNSNGNTYLYWFLQRPGQSPQLLIYRMS | 45 |
| | NLASGVPDRFSGSGSGTAFTLRISRVEAEDVGVYYCMQHLEYPYTFGGGTKLEIK | |
| HFB11-21 | DIVLTQSPASLAVSLGQRATISCRASESVDNYGISFMHWYQQKPGQPPRLLIYRASN | 47 |
| | LASGIPARFSGSGSSTDFTLTINPVETDDIATYYCQQSNKDPRTFGGGTKLEIK | |

TABLE 3
CDR regions 1-3 of heavy chain for anti-CCR8 mAbs as determined by
Kabat and IMGT method.

| | | SEQ | | SEQ | | SEQ |
|---|---|---|---|---|---|---|
| | | ID | | ID | | ID |
| VH | HC CDR1 | NO: | HC CDR2 | NO: | HC CDR3 | NO: |
| HFB11-10 | GFRFNTNA | 1 | IRSKSNNYATY | 2 | VRGSDNNYYYAMDY | 3 |
| HFB11-2 | GFSFNTYA | 13 | IRSKSNNYATY | 2 | VRGGSYYSNHYFDY | 14 |
| HFB11-3 | GFSFNTYA | 13 | IRSKSNNYATY | 2 | VRGKDANYFYAMDY | 15 |
| HFB11-5 | GFSFNAYA | 16 | IRTKSNNYATY | 17 | VRGGYGNGGYYFDY | 18 |
| HFB11-8 | GFRFNTNA | 19 | IRSKSNNYATH | 20 | VRGSDNNYYYAMDY | 21 |
| HFB11-19 | GYTFTDYI | 22 | FSPRNNYVK | 23 | SRHGRNSIGFAY | 24 |
| HFB11-21 | GYTFISYW | 25 | IDPNSGKTK | 26 | AREGWGDY | 27 |

TABLE 4
CDR regions 1-3 of light chain for anti-CCR8 mAbs as determined by
Kabat and IMGT method.

| | | SEQ | | SEQ | | SEQ |
|---|---|---|---|---|---|---|
| | | ID | | ID | | ID |
| VL | VL CDR1 | NO: | VL CDR2 | NO: | VL CDR3 | NO: |
| HFB11-10 | KSLLHSNGNTY | 4 | YRMSNL | 5 | MQHLEYPFT | 6 |
| HFB11-2 | KSLLHSNGNTY | 4 | YQMSNL | 28 | MQHLEYPFT | 6 |
| HFB11-3 | KSLLHSNGNTY | 4 | YRMSNL | 5 | MQHLEYPFT | 6 |
| HFB11-5 | KSLLHRNGNSY | 29 | YRISNL | 30 | MQHLEYPFT | 6 |
| HFB11-8 | KSLLHSNGNTY | 4 | YRMSNL | 5 | MQHLEYPFT | 6 |
| HFB11-19 | KSLLNSNGNTY | 31 | YRMSNL | 5 | MQHLEYPYT | 32 |
| HFB11-21 | ESVDNYGISF | 33 | YRASNL | 34 | QQSNKDPRT | 35 |

### Example 2: Anti-CCR8 Monoclonal Antibodies Bind Specifically to CCR8 Expressing Cells

Briefly, purified antibodies (range from 30 mM in PBS, 3-fold dilution) were incubated with 1×10⁵ stable CCR8 expressing-CHOK1 cells (Perkin Elmer; Waltham, MA) for 30 minutes at 4° C. After washing in MACS buffer (Miltenyi; Bergisch Gladbach, Germany), secondary anti-human-AF647 antibodies (Jackson ImmunoResearch; West Grove, PA) were incubated at 12 nM for 30 minutes in PBS. After washing in MACS buffer, cell pellets were resuspended in 50 μl PBS and samples were analyzed on iQue cytometer (Sartorious; Goettingen, Germany) using Forecyt software (Intellicyt; Ann Arbor, MI). Mean of fluorescence for AF647 channel from single cells are shown in FIG. 1.

There was no signal detected on parental CHOK1 cells; therefore, the tested antibodies were specific to hCCR8 (FIG. 1B). The EC₅₀ values obtained from the titration curve shown in FIG. 1A ranged from 0.5 to 9 nM (FIG. 1C).

### Example 3: Anti-CCR8 Monoclonal Antibody Recognized the N-Terminus, as Well as the Loop 1, which is Involved in Protein Conformation

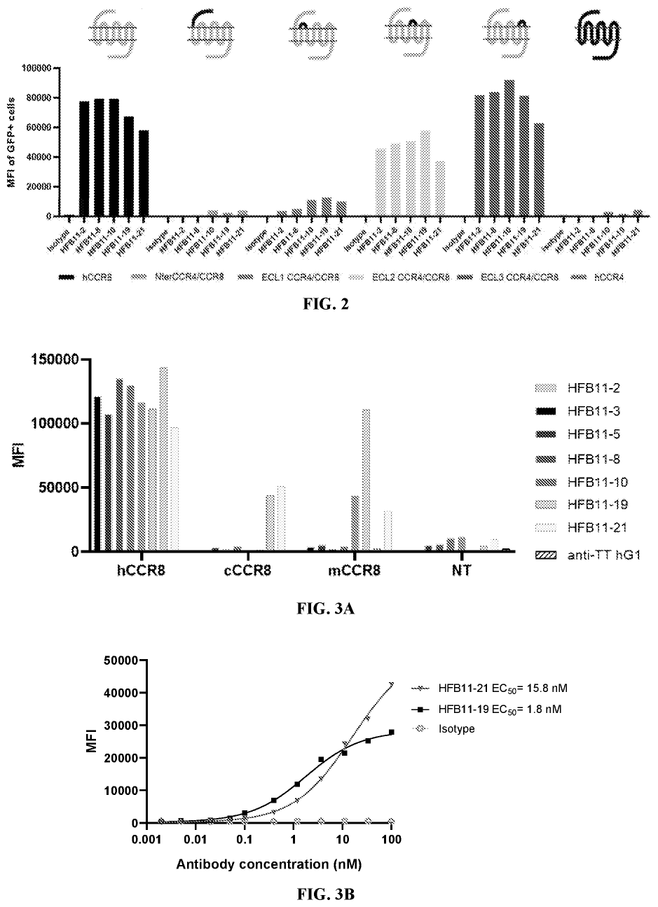

To map the epitope of CCR8, constructs expressing chimeric proteins containing portions of human CCR8 (Uniprot/Swiss-Prot: P51685 (SEQ ID NO:11)) fused to the closest chemokine receptor CCR4 (UniProt/Swiss-Prot: P51679 (SEQ ID NO:12)) were generated. The resultant proteins contain an N-terminal domain (amino acids 1-40 of SEQ ID NO:12), extracellular domain 1 ((ECL1) amino acids 100-112 of SEQ ID NO:12), extracellular domain 2 ((ECL2) amino acids 177-205 of SEQ ID NO:12), or extracellular domain 3 ((ECL3) amino acids 269-286 of SEQ ID NO:12) from human CCR4 with the remaining sequence from human CCR8 (SEQ ID NO:11). The constructs were made in pcDNA3.1 vector containing a cleavable intracellular GFP tag, and the constructs were expressed transiently at the surface of ExpiHEK293 cells. Purified antibodies (50 nM) were incubated with 1×10⁵ of ExpiHEK293 cell transiently transfected with either human, mouse, or cynoCCR8-GFP protein, for 30 minutes at 4° C. After washing, secondary anti-human-AF647 antibody (Jackson ImmunoResearch; West Grove, PA) was incubated at 12 nM for 30 minutes in PBS. After washing, cell pellets were resuspended in 50 μL PBS and samples were analyzed on iQue cytometer (Sartorious, Goettingen, Germany) using Forecyt software (Intellicyt, Ann Arbor, MI). Mean of fluorescence intensity for the specific signal are represented among GFP+ cells in FIG. 2.

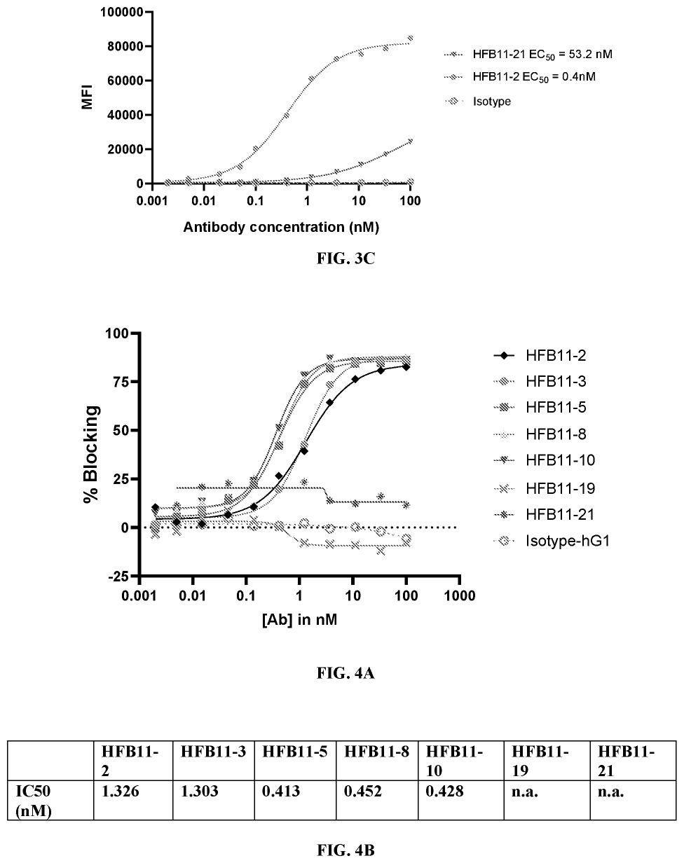

All antibodies were specific to human CCR8 and did not cross react with the closest family member, CCR4. The antibodies showed similar profiles on the CCR8/CCR4 mutants. When the N-terminal domain was replaced by CCR4, anti-CCR8 antibody lost reactivity. Binding activity strongly decreased upon replacement of extracellular domain 1 (ECL1). However, as the majority of the antibodies did not cross react with the cynoCCR8 (FIG. 3) and the ECL1 is 100% identical to the one in human CCR8, it suggested that the ECL1 loop played a critical role in protein conformation but may not be the epitope for the antibody.

As there was no impact with replacement of ECL2 and 3, it was determined that the anti-CCR8 antibodies generated comprised epitopes located in the N-terminal domain.

### Example 4: Anti-CCR8 Monoclonal Antibodies were Specific to Human CCR8 Protein and can Cross-React with the Mouse and/or Cyno CCR8 Receptors

Briefly, purified antibodies (saturating concentration of 50 nM, FIG. 3A or concentration range from 100 nM, 3-fold dilution, FIGS. 3B-3C) were incubated with 1×10⁵ ExpiHEK293 cells transiently transfected with either human (SEQ ID NO:11; Uniprot/Swiss-Prot: P51685), mouse CCR8 (SEQ ID NO: 64; Uniprot/Swiss-Prot: P56484), or cynoCCR8 (SEQ ID NO: 65; Uniprot/Swiss-Prot: G7NYJ2) fused to GFP protein via cleavable linker, for 30 minutes at 4° C. After washing, secondary anti-human-AF647 antibody (Jackson ImmunoResearch; West Grove, PA) was incubated at 12 nM for 30 minutes in PBS. After washing, cell pellets were resuspended in 50 μL PBS and samples were analyzed by flow cytometry on iQue cytometer (Sartorious, Goettingen, Germany) using Forecyt software (Intellicyt, Ann Arbor, MI). Mean of fluorescence intensity for the specific signal among GFP+ cells are represented in FIGS. 3A-3C. All antibodies tested at saturating concentration are shown in FIG. 3A. Titration curve of cross-reactive antibodies against mouse CCR8 and cyno CCR8 are presented in FIGS. 3B and 3C, respectively.

Different cross reactivity profiles were observed among the anti-CCR8 antibodies: one main group, comprising HFB11-3, HFB11-5, HFB11-8 and HFB11-10 were only specific to the human protein and did not cross-react with other species. HFB11-2 was cross reactive to the mouseCCR8 protein; HFB11-19 recognized the cynoCCR8 protein; and HFB11-21 was able to bind to both mouse and cyno CCR8 proteins. It is believed that these antibodies recognize different epitopes among the N-terminal domain. The titration curve of the mouse CCR8 antibodies demonstrated similar binding properties for HFB11-2 with sub-nanomlar EC₅₀ but lower binding potency for HFB11-21 with EC₅% of 53 nM (FIG. 3B). On the other hand, this antibody demonstrated higher binding properties to the cyno CCR8 receptor with EC₅₀ of 15.8 nM. HFB11-19 is a more potent binder to cyno CCR8 with an EC₅₀ of 1.8 nM (FIG. 3C).

### Example 5: Anti-CCR8 Antibody Blocks CCL1 Binding to CCR8 Expressing Cells

The capacity to block CCL1 binding to human CCR8 expressing cells was assessed. Anti-CCR8 antibody, at several concentrations (dilution range starting from 100 nM) was added to stable CCR8-expressing cells (CHOK1, Perkin Elmer) for 30 minutes at 4° C. After washing in MACS buffer, hCCL1-AF647 at 30 nM (Almac) was added and incubated at 4° C. for 45 minutes. After washing in MACS buffer, the pelleted cells were resuspended in 50 μL before reading by flow cytometry using an iQue cytometer (Sartorious, Goettingen, Germany) and Forecyt software (Intellicyt, Ann Arbor, MI). Maximum mean fluorescence intensity (MFI) was obtained without antibody to reflect 100% of CCL1 binding. MFI for AF647 channel was used to calculate the % CCL1 blocking (formula=100−(MFIₛₐₘₚₗₑ*100/MFI_{CCL1 alone})). FIG. 4 shows the results demonstrating that 5/7 anti-CCR8 antibodies block CCL1 binding to CCR8.

HFB11-19 and HFB-21, that were both cyno cross reactive, were not able to block hCCL1 binding to hCCR8 expressed at the cell surface, whereas, all the other antibodies efficiently blocked hCCL1 with IC₅₀ values ranging from ˜0.4 to 1.4 nM (FIG. 4B). This suggests that HFB11-19 and HFB11-21 share a different epitope compared to the other antibodies on the N-terminal domain.

### Example 6: Anti-CCR8 Antibodies Inhibit Intracellular CA²⁺ Flux

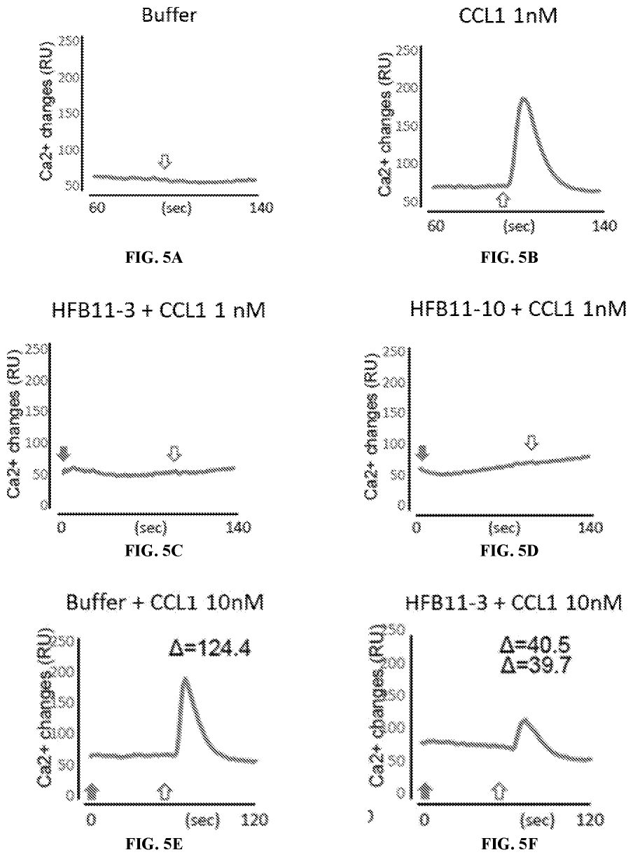

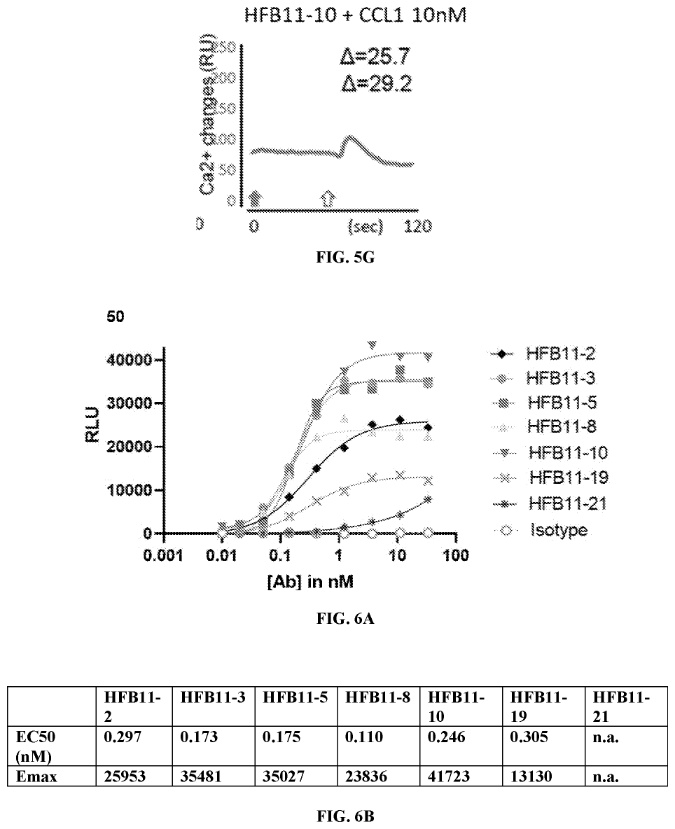

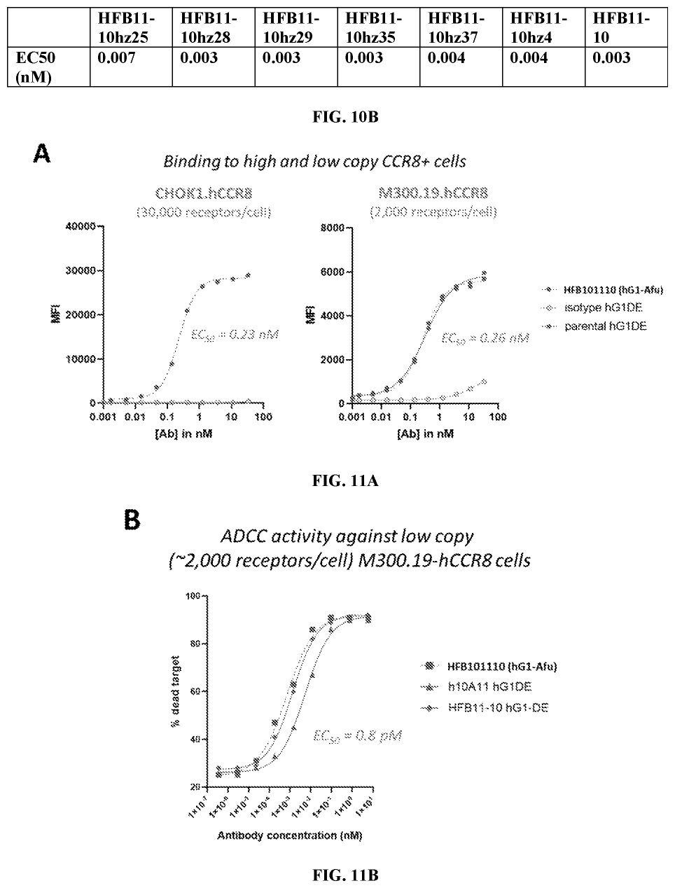

CCR8 low copy cells (M300.19 mouse pre-B cells) were treated at time 0 seconds with 100 nM of HFB11-10 antibody (grey arrow, FIGS. 5D and 5G), HFB11-3 antibodies (grey arrow, FIG. 5 or buffer (FIG. 5E)), followed by incubation for 90 seconds and an addition of 1 nM of CCL1 (clear arrows, FIGS. 5B-5D) or 10 nM of CCL1 (clear arrows, FIGS. 5E-5G). Negative control with buffer only is shown in FIG. 5A (clear arrows). FIGS. 5C and 5D demonstrate that both antibodies interfered with CCL1-induced Ca²⁺ spikes in CCR8-M300.19 cells, with complete signal inhibition at 1 nM CCL. With increased CCL1 concentration to 10 nM partial inhibition of 61 and 77.5% for HFB11-3 and HFB11-10 was observed, respectively (FIGS. 5F and 5G). It was also demonstrated that anti-CCR8 antibodies that blocked CCL1 binding also blocked the intracellular signaling induced by the chemokine.

### Example 7: Anti-CCR8 Antibodies were Able to Engage CD16 in Antibody Dependent Cellular Cytotoxicity (ADCC) Reporter Bioassay

CCR8 expressing CHOK1 cells (Perkin Elmer) were used as target cells in the ADCC reporter Bioassay (Promega). Briefly, anti-CCR8 antibodies (range from 30 nM, 3-fold dilution), target cells (25000/well) were co-cultured with Jurkat engineered cells expressing CD16 and luciferase reporter gene, at E/T=3:1. After 6 hours of incubation at 37° C., 5% CO₂, Bio-glo reagent was added and incubated for 5 minutes at room temperature before reading the bioluminescence using a Tecan plate reader. The RLU signals are shown in the graph (FIG. 6A).

All anti-CCR8 antibodies were able to engage CD16 with a variable efficacy. HFB11-19 and HFB11-21, that are the non-blocking antibodies showed lower potency (lower Eₘₐₓ and EC₅₀) compared to the other antibodies. HFB11-2, HFB11-3, HFB11-5, HFB11-8 and HFB11-10 showed high potency with EC₅₀ values ranging from 0.1 to 0.3 nM and an Eₘₐₓ between >25000 and >40000 RLU (FIG. 6B).

### Example 8: Humanized Anti-CCR8 Antibodies Strongly Bind to CCR8 Expressing Cells

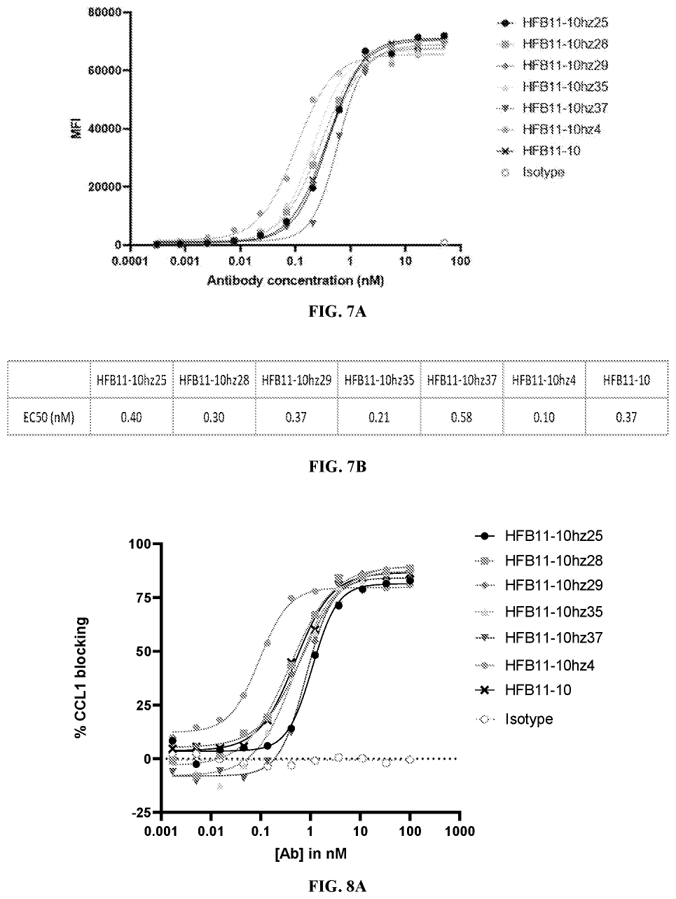

Purified antibodies (produced in an ADCC-enhanced format) were incubated with 1×10⁵ hCCR8-CHOK1 stable cells or parental CHOK1 cells (range from 50 nM in MACS buffer, 3-fold dilution) for 30 minutes at 4° C. After washing, secondary anti-human-AF647 antibody was incubated at 12 nM for 30 minutes in MACS buffer. After washings, cell pellets were resuspended in 50 μL MACS buffer and samples were analyzed by cytometry on iQue cytometer (Sartorious, Goettingen, Germany) using Forecyt software (Intellicyt, Ann Arbor, MI). Mean of florescence for AF647channel among single cell gated are represented in FIG. 7.

Titration curves showed that the humanized anti-CCR8 antibody candidates were all able to bind CCR8 expressing cells with a very low EC₅₀ of 0.40, 0.30, 0.37, 0.21, 0.58 and 0.10 nM for the humanized variants Hz25, HZ28, Hz29, Hz35, Hz37 and Hz4, respectively, without background of the isotype antibody (FIGS. 7A and 7B).

Humanized antibody sequences are provided below:

TABLE 5
Sequences of humanized heavy chain variable regions for H11-10 mAb.

| | | SEQ |
|---|---|---|
| | | ID |
| VH | VH sequence | NO: |
| H11-10 VH10 | EVQLVESGGGLVQPGGSLKLSCAASGFRFNTNAMNWVRQASGKDLEWVARIRS | 9 |
| | KSNSYATYYAASVKGRFTISRDDSKNTLYLQMNSLKTEDTAVYYCTRGSDNNY | |
| | YYAMDYWGQGTTVTVSS | |
| H11-10 VH1 | EVQLVESGGGLVQPGGSLKLSCAASGFRFNTNAMNWVRQASGKGLEWVGRIRS | 52 |
| | KSNNYATYYAASVKGRFTISRDDSKNTAYLQMNSLKTEDTAVYYCTRGSDNNY | |
| | YYAMDYWGQGTTVTVSS | |
| H11-10 VH2 | EVQLVESGGGLVQPGGSLKLSCAASGFRFNTNAMNWVRQASGKDLEWVARIRS | 53 |
| | KSNNYATYYAASVKGRFTISRDDSKNTLYLQMNSLKTEDTAVYYCTRGSDNNY | |
| | YYAMDYWGQGTTVTVSS | |
| H11-10 VH3 | QVQLVESGGGVVQPGRSLRLSCAASGFRFNTNAMNWVRQAPGKGLEWVARIRS | 54 |
| | KSNNYATYYADSVKGRFTISRDNSKNTLYLQMNSLRAEDTAVYYCVRGSDNNY | |
| | YYAMDYWGQGTTVTVSS | |
| H11-10 VH4 | EVQLVESGGGLVQPGRSLRLSCTASGFRFNTNAMNWFRQAPGKGLEWVARIRS | 55 |
| | KSNNYATYYTASVKGRFTISRDGSKSIAYLQMNSLKTEDTAVYYCVRGSDNNY | |
| | YYAMDYWGQGTTVTVSS | |
| H11-10 VH5 | QVQLVQSGAEVKKPGASVKVSCKASGFRFNTNAMNWVRQAPGQDLEWVARIR | 56 |
| | SKSNNYATYYSQKFQGRVTITRDTSASTAYMELSSLRSEDTAVYYCVRGSDNNY | |
| | YYAMDYWGQGTTVTVSS | |
| H11-10 VH6 | QVQLQESGPGLVKPSGTLSLTCAASGFRFNTNAMNWVRQPPGKGLEWVGRIRS | 57 |
| | KSNNYATYYNPSLKSRVTISRDKSKNQFSLKLSSVTAADTAVYYCVRGSDNNYY | |
| | YAMDYWGQGTTVTVSS | |
| H11-10 VH8 | QVTLRESGPALVKPTQTLTLTCTASGFRFNTNAMNWVRQPPGKALEWVARIRSK | 58 |
| | SNNYATYYSTSVKTRLTISKDTSKNQLVLTMTNMDPVDTATYYCVRGSDNNYY | |
| | YAMDYWGQGTTVTVSS | |
| H11-10 VH9 | EVQLVESGGGLVQPGGSLKLSCAASGFRFNTNAMNWVRQASGKGLEWVGRIRS | 59 |
| | KSNSYATYYAASVKGRFTISRDDSKNTAYLQMNSLKTEDTAVYYCTRGSDNNY | |
| | YYAMDYWGQGTTVTVSS | |
| H11-10 VH11 | QVQLVESGGGVVQPGGSLRLSCAASGFRFNTNAMNWVRQAPGKDLEWVARIR | 60 |
| | SKSNNYATYYADSVKGRFTISRDNSKNTLYLQMNSLRAEDTAVYYCVRGSDNN | |
| | YYYAMDYWGQGTTVTVSS | |

TABLE 6
Sequences of humanized light chain variable regions for H11-10 mAb.

| | | SEQ |
|---|---|---|
| | | ID |
| VL | VL sequence | NO: |
| H11-10 VL1 | DIVMTQSPLSLPVTPGEPASISCRSSQSLLHSNGNTYLYWFLQKPGQSPQLLIYRMS | 10 |
| | NRASGVPDRFSGSGSGTDFTLKISRVEAEDVGVYYCMQHLEYPFTFGQGTKLEIK | |
| H11-10 VL2 | EIVMTQTPLSLSVTPGEQASISCRSSKSLLHSNGNTYLYWFLQKARPSSTLLIYRMS | 61 |
| | NLASGVPDRFSGSGSGTDFTLKISRVEAEDVGVYYCMQHLEYPFTFGQGTKLEIK | |
| H11-10 VL3 | DIQMTQSPSAMSVSVGDRVTITCRSSKSLLHSNGNTYLYWFQQKPGKVPKLLIYR | 62 |
| | MSNLASGVPSRFSGSGSGTEFTLTISSVQPEDVATYYCMQHLEYPFTFGQGTKLEIK | |
| H11-10 VL4 | EIVLTQSPGTLSLSPGERATLSCRSSKSLLHSNGNTYLYWFQQKPGQAPRLLIYRM | 63 |
| | SNLASGIPDRFSGSGSGTDFTLTISRLEPEDVAVYYCMQHLEYPFTFGQGTKLEIK | |

TABLE 7
CDR regions 1-3 of humanized heavy chain for humanized anti-CCR8 mAbs
as determined by Kabat and IMGT method.

| | | SEQ | | SEQ | | SEQ |
|---|---|---|---|---|---|---|
| | | ID | | ID | | ID |
| VH | HC CDR1 | NO: | HC CDR2 | NO: | HC CDR3 | NO: |
| H11-10 VH10 | GFRFNTNA | 1 | IRSKSNSYATY | 48 | TRGSDNNYYYAMDY | 49 |
| H11-10 VH1 | GFRFNTNA | 1 | IRSKSNNYATY | 2 | TRGSDNNYYYAMDY | 49 |
| H11-10 VH2 | GFRFNTNA | 1 | IRSKSNNYATY | 2 | TRGSDNNYYYAMDY | 49 |
| H11-10 VH3 | GFRFNTNA | 1 | IRSKSNNYATY | 2 | VRGSDNNYYYAMDY | 3 |
| H11-10 VH4 | GFRFNTNA | 1 | IRSKSNNYATY | 2 | VRGSDNNYYYAMDY | 3 |
| H11-10 VH5 | GFRFNTNA | 1 | IRSKSNNYATY | 2 | VRGSDNNYYYAMDY | 3 |
| H11-10 VH6 | GFRFNTNA | 1 | IRSKSNNYATY | 2 | VRGSDNNYYYAMDY | 3 |
| H11-10 VH8 | GFRFNTNA | 1 | IRSKSNNYATY | 2 | VRGSDNNYYYAMDY | 3 |
| H11-10 VH9 | GFRFNTNA | 1 | IRSKSNSYATY | 48 | TRGSDNNYYYAMDY | 49 |
| H11-10 VH11 | GFRFNTNA | 1 | IRSKSNNYATY | 2 | VRGSDNNYYYAMDY | 3 |

TABLE 8
CDR regions 1-3 of humanized light chain for humanized anti-CCR8 mAbs
as determined by Kabat and IMGT method.

| | | SEQ | | SEQ | | SEQ |
|---|---|---|---|---|---|---|
| | | ID | | ID | | ID |
| VL | VL CDR1 | NO: | VL CDR2 | NO: | VL CDR3 | NO: |
| H11-10 VL1 | QSLLHSNGNTY | 50 | YRMSNR | 51 | MQHLEYPFT | 6 |
| H11-10 VL2 | KSLLHSNGNTY | 4 | YRMSNL | 5 | MQHLEYPFT | 6 |
| H11-10 VL3 | KSLLHSNGNTY | 4 | YRMSNL | 5 | MQHLEYPFT | 6 |
| H11-10 VL4 | KSLLHSNGNTY | 4 | YRMSNL | 5 | MQHLEYPFT | 6 |

### Example 9: Humanized Anti-CCR8 Antibodies Blocked CCL1 Binding to CCR8

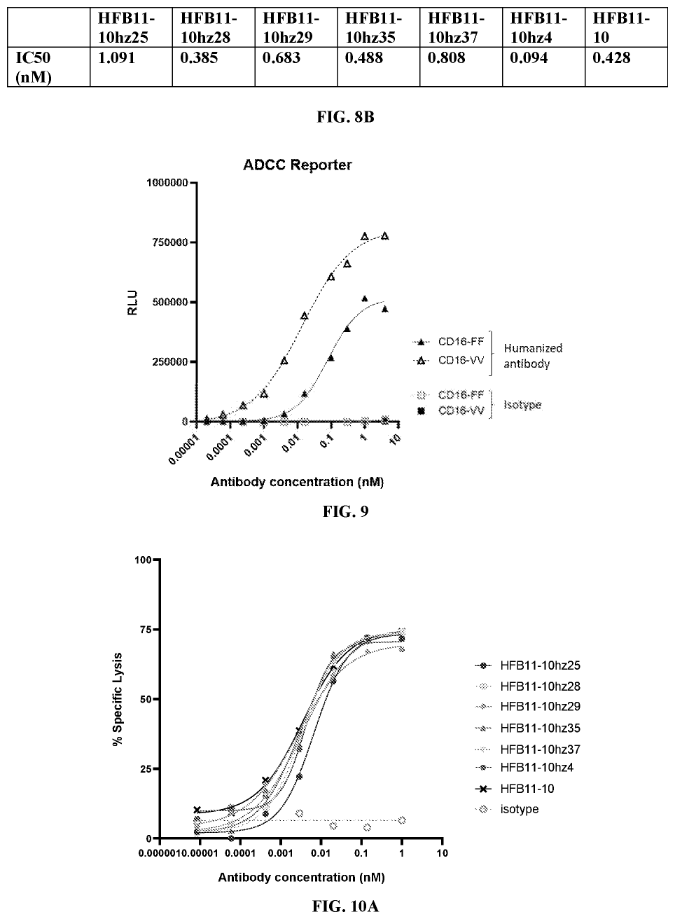

1×10⁵ stable CHOK1.hCCR8 cells (Perkin Elmer) were incubated with a range of the humanized anti-CCR8 antibodies (produced in an ADCC enhanced format) or an isotype antibody (concentration range from 33 nM, 3-fold dilution) for 30 minutes at 4° C. After centrifugation for 3 minutes at 300 g, the cells were resuspended with 30 nM of human CCL1 ligand labeled with AF647 (50 μL) (Almac) for a 45-minute incubation at 4° C. After 2 washes, the cells were analyzed by flow cytometry using an iQue cytometer (Sartorious, Goettingen, Germany) using Forecyt software (Intellicyt, Ann Arbor, MI). % blocking was calculated as described in Example 5 and presented in FIG. 8.

The humanized anti-CCR8 antibodies in the ADCC enhanced format were able to block human CCL1 binding to CCR8 expressing cells with a potent IC₅₀ of 0.09, 1.09, 0.39, 0.68, 0.49 and 0.81 nM for HFB11-10 Hz4, HFB11-10 Hz25, HFB11-10 Hz28, HFB11-10 Hz29, HFB11-10 Hz35 and HFB11-10 Hz37 antibodies, respectively (FIG. 8).

### Example 10: Humanized HFB11-10 Hz37 Anti-CCR8 Antibody Engaged CD16 F and V Allotypes on an ADCC Reporter Assay

Stable hCCR8-CHOK1 cells (Perkin Elmer) were used as target cells and co-cultured with engineered stable Jurkat cells expressing CD16 (FF or VV phenotype) and reporter NFAT gene from ADCC report Bioassay kit at an E/T ratio of 3:1 (Promega). A range of HFB11-10 Hz37antibody was also added to the culture from 30 nM (3-fold serial dilution). After 6 hours of incubation at 37° C. and 5% CO₂, BioGlo luciferase substrate was added following supplier recommendation and luminescence signal was read after an incubation for 5 minutes using a Tecan plate reader. Bioluminescence signal is reporter as RLU in the graph (FIG. 9).

Humanized anti-hCCR8 antibody was able to engage both CD16 F and V allotypes in an ADCC reporter assay mediating target cell killing. As expected, a higher potency with the high affinity Fcγ receptor (VV allotype) was observed with an EC₅₀ of 0.014 nM, that increased to 0.080 nM with the low affinity Fcγ receptor (FF allotype) (FIG. 9).

### Example 11: Humanized Anti-CCR8 Antibody Mediated ADCC on Cells Expressing Low Copies of CCR8

NK cells were isolated using an NK cell isolation kit (Miltenyi) from healthy human PBMCs. Two million NK cells per well in a 24-well plate were activated in IL2 RPMI media at 37° C., 5% CO₂ for about 30 hours.

Cells expressing a low copy number of CCR8 (M300.19-mouse pre-B cells stable transfected) were labeled with Celltrace™ FarRed (Invitrogen; Waltham, MA) before co-culturing with NK cells (E/T ration 2:1) and a range of HFB11-10 antibody concentrations (humanized and non-humanized in ADCC enhanced format) for 16 hours at 37° C. in U-bottom 96-well plates. Dead cells were labeled with Nucgreen before reading the fluorescence using the cytoflex cytometer (Beckman Coulter). The percentage of dead target cells was assessed as the double positive cells and the percentage of specific target cell lysis was assessed as (% target dead cell with antibodies−% target dead cell without antibodies)×100/(100−% target dead cells without antibodies). Spontaneous cell death was assessed in the condition target cells co culture with NK cells without antibodies.

FIG. 10 shows the specific ADCC NK killing mediated by the humanized anti-CCR8 antibodies. Humanized variants and the parental antibody (in ADCC enhanced format) demonstrated high potency to mediate ADCC NK killing of CCR8 expressing low copy cells in the assay (E:T ratio of 3:1). The EC₅₀ was low, ranging from 0.003 nM (for the Hz29, Hz35, parental Ab) to 0.007 nM for the Hz25 antibody (mean from n=3 donors). All humanized antibodies demonstrated very potent activity in ADCC assay using primary NK cells.

### Example 12: In Vitro Characterization of Humanized Anti-CCR8 Antibody HFB101110

Afucosylated humanized anti-CCR8 antibody HFB101110, which is related to HFB11-10, was further studied. In the following examples, anti-CCR8 antibodies HFB101110 and HFB11-10 may be used alternatively.

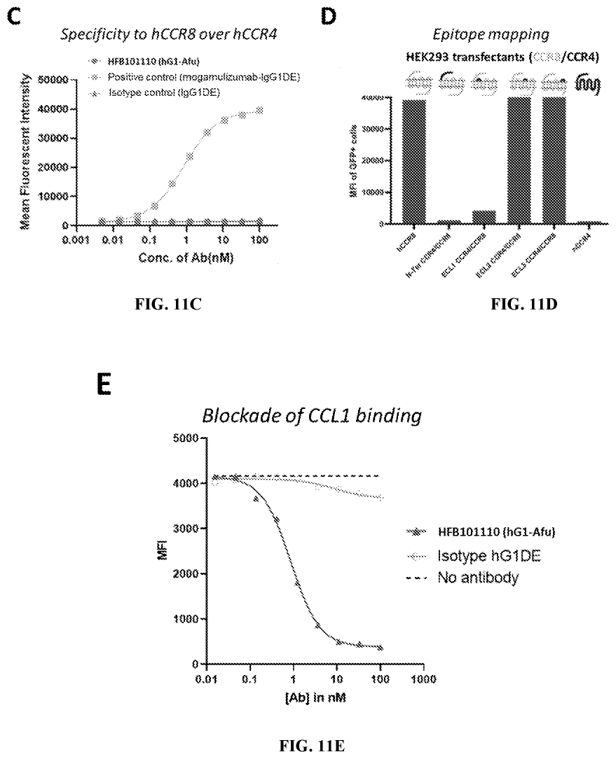

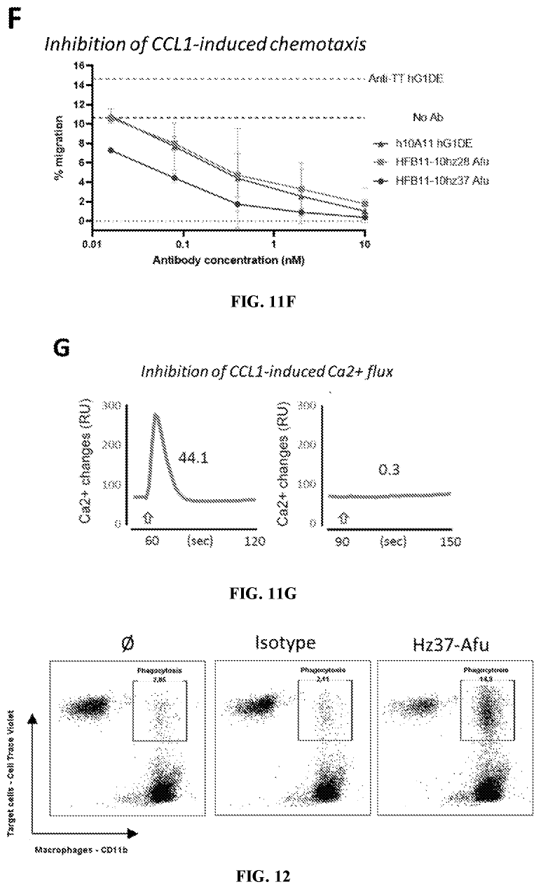

As illustrated in FIGS. 11A-11G, anti-CCR8 mAb HFB101110 was assessed in further details. It was confirmed that the antibody had a strong binding to CCR8 on both high-copy (CHOK1-hCCR8, ˜30,000 receptors/cell, left) and low-copy (M300.19-hCCR8, ˜2,000 receptors/cell, right) cells as measured by flow cytometry, with an EC₅₀ of 0.23 nM and 0.26 nM, respectively (FIG. 11A). The parental antibody to HFB101110 formatted as an hIgG1 antibody with normal glycosylation and DE mutations in the Fc region to enhance ADCC activity (HFB101110-hIgG1-DE) was used as a positive control for afucosylated HFB101110. HFB101110 was able to bind both high- and low-copy hCCR8-expressing cells with similar sub-nM EC₅₀ values.

Anti-CCR8 mAb HFB101110 induced ADCC activity against M300.19-hCCR8 cells. Exogenous primary NK cells isolated from PBMCs were added at an Effector:Target ratio of 3:1, and dead cells were quantified by flow cytometry using NucGreen staining after 16 h of co-culture. Mean values of experiments using NK cells from 3 different donors are shown. HFB101110-hIgG1-DE and another anti-hCCR8 DE-formatted hIgG1 antibody were used as comparators (FIG. 11B).

The binding of HFB101110 to the related chemokine receptor CCR4 was evaluated by flow cytometry, with the anti-CCR4 antibody mogamulizumab used as a positive control. HFB101110 showed no binding to hCCR4, indicating specific recognition of hCCR8 by this antibody (FIG. 11C).

Domain swapping experiments were performed to identify the region of CCR8 recognized by HFB101110. Portions of hCCR8 were replaced with homologous domains of the related chemokine receptor CCR4, and the resulting chimeric proteins were expressed in 293 cells and binding of HFB101110 was assessed by flow cytometry (FIG. 11D). Based on these experiments, it was determined that HFB101110 recognizes the N-terminal extracellular domain of hCCR8.

As shown in FIG. 11E, blockade of hCCL1 binding to hCCR8 by HFB101110 was measured by flow cytometry. HFB101110-mediated blockade of chemotaxis of CCR8+ cells induced by recombinant hCCL1 was measured by a transwell migration assay. CCL1 was present at a concentration of 30 nM while varying the amount of antibody present. HFB101110-hIgG1-DE as well another humanized variant were used as comparators (FIG. 11F).

As shown in FIG. 11G, HFB101110-mediated blockade of calcium flux induced by the addition of hCCL1 to CCR8+ cells was evaluated. 10 nM hCCL1 was added to CCR8+ cells at the time indicated by the arrow, in the presence or absence of 10 μg/mL HFB101110.

### Example 13: Humanized Anti-CCR8 Antibody Demonstrated Antibody Dependent Cellular Phagocytosis (ADCP) Activity

Primary macrophages were isolated from human PBMCs after CD14+ bead isolation (Miltenyi). 2M cells/well were seed into 6-well plates with serum free RPMI 1640 medium for 1 hour at 37° C., 5% CO₂. The medium was then replaced with activating media (RPMI 1640, 10% FBS, 1% Penicillin/Streptomycin, 50 ng/mL M-CSF) and refreshed every 2 days for 1 week. IL13 was added to the activating media for the last 2 days of activation. Target cells (Stable hCCCR8.M300.19 cells) were labeled with Celltrace™ violet (Invitrogen) (20 minute incubation at 37° C., 5% CO₂). After washing in complete media, CCR8 cells were incubated 15 minutes at 37° C., 5% CO₂ with the humanized anti-CCR8 antibody at saturating concentration (100 nM), and then co-cultured with activated macrophages for four (4) hours. The macrophages were stained with anti-CD11b-APC antibody (Biolegend) before analysis by flow cytometry using Cytoflex cytometer (Beckman Coulter). Phagocytotic cells were identified as double positive cells using FlowJo software.

As shown in FIG. 12, about 15% of the cells were phagocytosed or under the process of phagocytosis by macrophages after the four (4)-hour incubation (FIG. 12). The humanized anti-CCR8 antibody HFB11-10 Hz37 specifically mediated CCR8+ cell phagocytosis through an ADCP mechanism using primary macrophages cells.

### Example 14: Humanized Anti-CCR8 Antibody Mediated Anti-Tumor Activity to MC38 Cells in hCCR8-KI Mice

### Mice and Tumor Cell Lines:

Female, 8-week old C57BL/6, mice were purchased from Biocytogen Jiangsu Co., Ltd (Jiangsu, China). The MC38 mouse colon carcinoma cell line was used for in vivo tumor efficacy studies (Biocytogen Jiangsu Co., Ltd.) The MC38 cell line was passaged twice prior to storage, thawed, and passaged twice prior to implantation for all described tumor experiments. The cells were determined to be free of Mycoplasma.

The study was performed following protocols reviewed and approved by the Institutional Animal Care and Use Committee (IACUC) of Biocytogen in accordance with the guidelines by the Association for Assessment and Accreditation of Laboratory Animal Care (AAALAC).

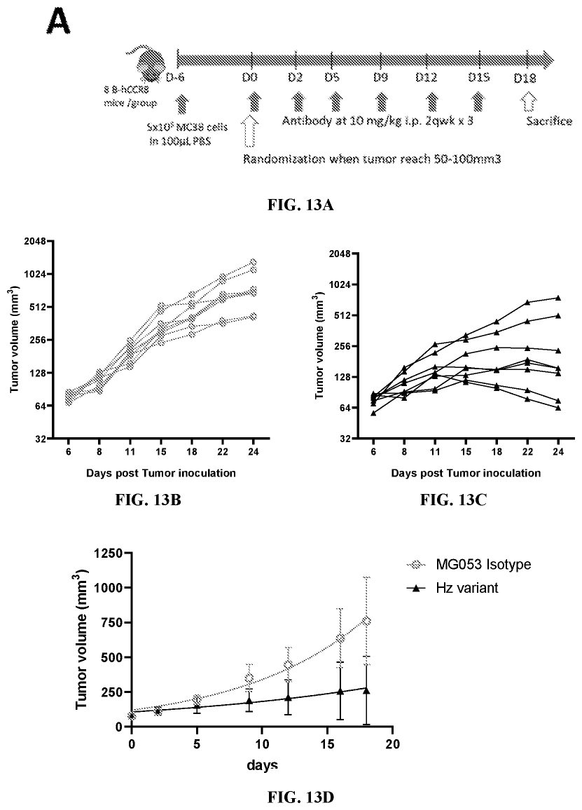

hCCR8 KI C57BL/6 mice (8 weeks old, 15-21 grams) were subcutaneously injected in the right front flank with MC38 tumor cells (5×10⁵) in 0.1 mL PBS. One week after tumor implantation, 10 mg/kg of humanized HFB11-10 or isotype antibody was injected intraperitoneally bi-weekly for 3 weeks, (n=8/group). Tumor volume (mm³) and body weight were measured twice per week (see FIG. 13A, which illustrates a schematic of in vivo efficacy study in hCCR8 knock-in mice). For this study, a non-afucosylated version of HFB101110 formatted as an mIgG2a antibody was used, to facilitate engagement with mouse Fc receptors.

FIGS. 13B-13D show the evaluation of anti-tumor activity by administration of humanized anti-CCR8 antibody (FIG. 13C-13D) or isotype mIgG2a (FIG. 13B-13D) using MC38 cells derived from colorectal cancers in the hCCR8-KI mice. Starting from day 9 post treatment initiation, the tumor growth was significantly reduced in the anti-human CCR8 treated group compared to the isotype control (p<0.01, **). Furthermore, in 3 of 8 mice tumor regression was observed from day 11 after tumor implantation. Collectively, these results demonstrated that anti-CCR8 antibody significantly reduced tumor growth.

### Example 15: Humanized Anti-CCR8 Antibody Therapy Reprogrammed the Tumor Microenvironment

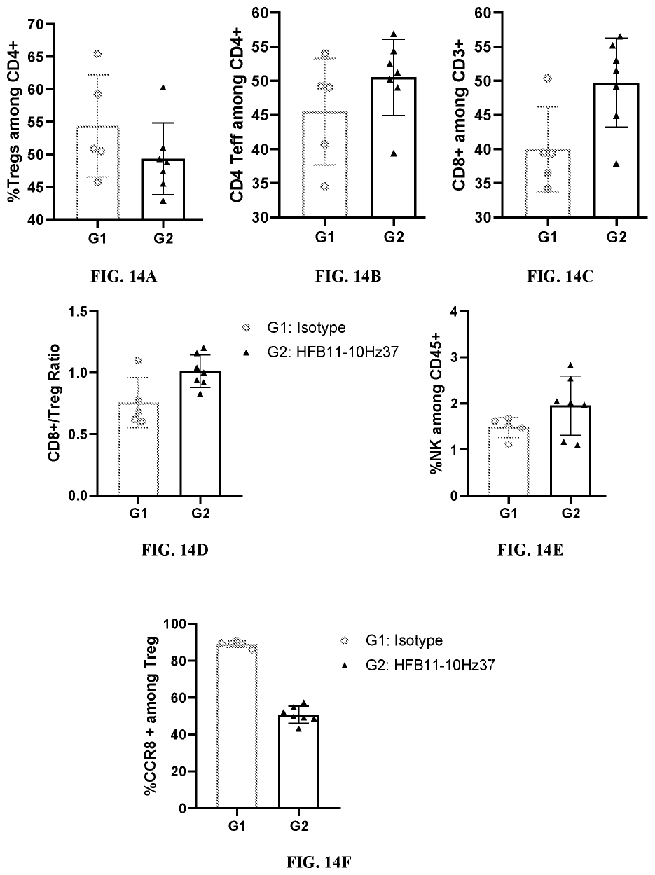

To better define the mechanism of action, the changes in the MC38 tumor microenvironment (TME) elicited by an anti-CCR8 antibody therapy was examined. As shown in FIGS. 14A-14F, anti-CCR8 antibody treatment altered the tumor infiltrating lymphocyte (TIL) composition in treated mice.

It was observed that anti-CCR8 antibody administration significantly increased the percentage of tumor infiltrating CD8+ (*, p value=0.0262, FIG. 14C). Moreover, a significant increase in the CD8/Tregs ratio was observed (*, p value=0.0240, FIG. 14D), further suggesting a tipping of the immune balance in favor of anti-tumor immunity. The treatment also slightly increased the percentage of NK cells and CD4+ effector T cells in the TME (FIGS. 14E and 14B, respectively).

Additionally, the anti-CCR8 antibody treatment did not significantly reduce Foxp3+CD4+ at the terminal endpoint (FIG. 14A), but the anti-CCR8 antibody treatment did significantly reduce the CCR8+ Treg population (FIG. 14F), demonstrating the target-specific effects of this agent. The decrease of 58% CCR8+ Tregs observed compared to the isotype group reflected that the target occupancy by the humanized antibody used in the FACs panel was competing for the same epitope.

### Example 16: CCR8 Expression in T-Cell Populations from Human Primary Tumors from Renal Cell Cancer (RCC) Patients and from Lung Cancer Patients

Fresh tumor infiltrating lymphocytes were digested and dissociated into single cell suspension, counted and frozen down before analysis. Briefly, the gentle MACS tumor dissociation kit (Miltenyi) was first used. Tumors were cut in small pieces and incubated with enzyme mix and tissue was digested using gentle MACS (Miltenyi). Samples were then centrifuged at 300 g for 5 minutes and washed twice with 10 mL of cold media. Samples were then filtered under 30 μM and resuspended in cold freezing media (90% FBS and 10% DMSO) before further analysis. One aliquot was thawed and incubated with the antibody panel for CCR8 phenotyping. The sample was first stained with live/dead dye and FcBlock. Then, extracellular staining with anti-CD45, anti-CD3, anti-CD4, anti-CD8, anti-CD56, anti-TIGIT antibodies (Biolegend) and anti-CCR8 (BD) was performed. Finally, samples were permeabilized and stained with FoxP3 for intracellular staining. Samples were analyzed by flow cytometry using FLowJo software.

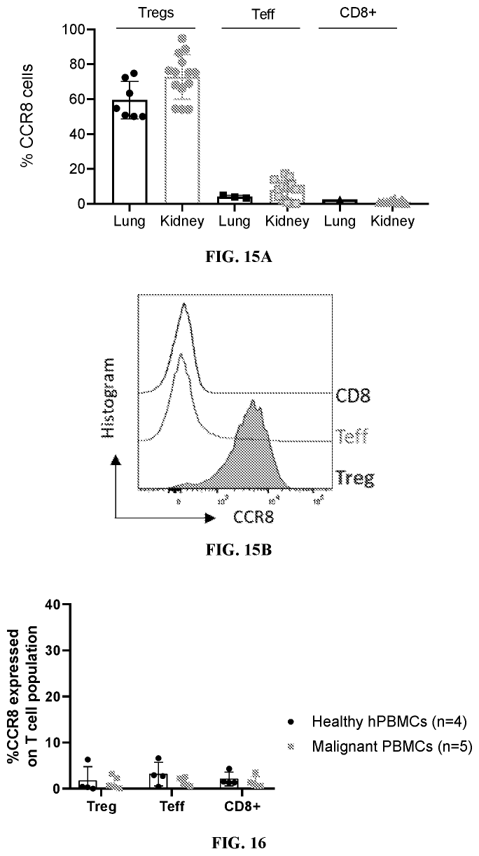

CCR8 expression was observed on Tregs from 55-95% of Renal Cell Carcinoma patients (n=13), with a mean of 72.8=12.8% (FIG. 15A). Conversely, CCR8 expression on FoxP3-CD4+T effector cells was less than 20% (mean of 7.9=5.6%) and close to zero for CD8+ T cells (mean of 1.2=0.8%, FIG. 14A). In the histogram shown in FIG. 15B, the cytometry data was reported for donor #726, where 96% of Tregs decreased. CCR8 was confirmed to be a target of interest for the depletion of Tregs without affecting other T cell populations within RCC tumors.

### Example 17: CCR8 Expression in T-Cell Populations from the Circulation in Healthy and Malignant PBMCs

CCR8 phenotyping was performed as described in the example 15. Briefly, PBMCs from healthy (n=4) or malignant (n=5) donors were isolated from fresh blood. ⅓ volume of Ficoll was added to the blood sample followed by and centrifugation at 2000 rpm for 30 minutes at RT. The White ring containing immune cells were collected and washed twice with PBS by centrifugation at 1250 rpm for 10 minutes at RT before FACS analysis. Percentage of CCR8+ among T cell population is presented FIG. 16.

CCR8 expression observed in the circulation of healthy or malignant patient was very low (<4%) for all T-cell populations (FIG. 16). There are no differences between Tregs or Teff and CD8+ T cells. This analysis confirms the specificity of the target that was restricted to tumor site and we do not expect side effect in the circulation, limiting risk of toxicity and autoimmunity.

### Example 18: Humanized Anti-CCR8 Antibody Mediated ADCC NK Killing Activity on Primary Human Tumor Infiltrating Lymphocytes (TILS) from RCC Patients

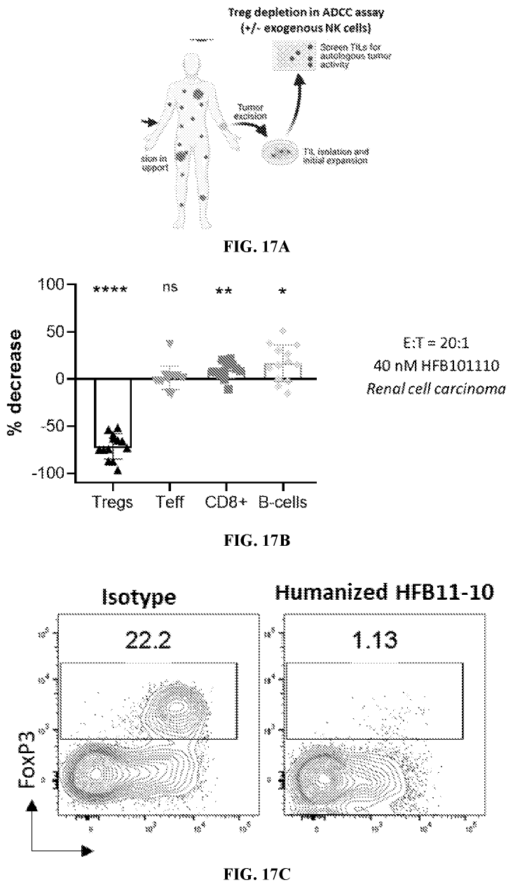

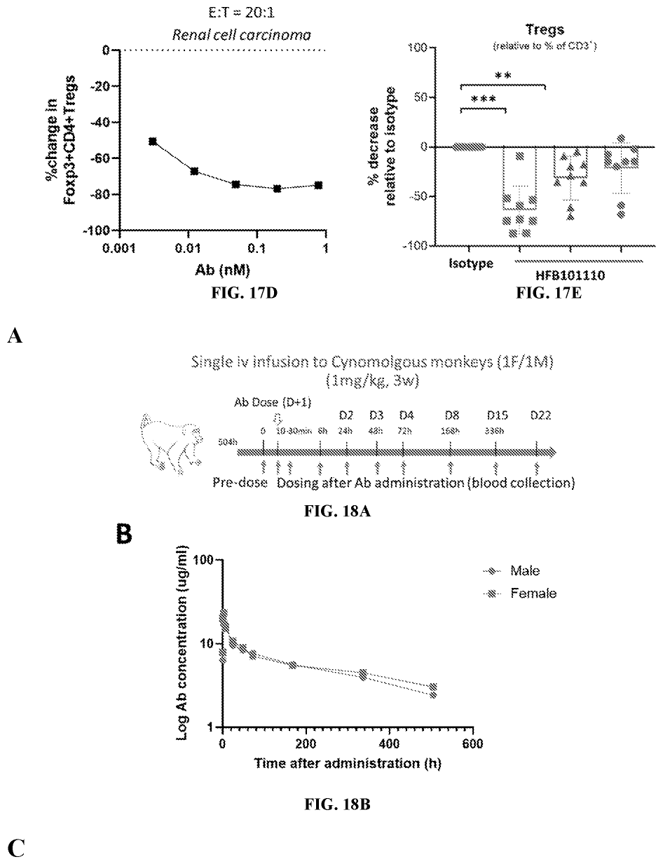

FIG. 17A illustrates a schematic of sample collection and ADCC assay as performed for ex vivo killing of primary tumor-infiltrating lymphocytes mediated by HFB101110. Single cell suspensions of Tumor infiltrating lymphocytes (TIL) from RCC patients (n=13) were thawed and the viability were assessed using AO/PI fluorescence on a countess II device. TILs were split into 100 μL/well of a 96-well U-bottom plates for the ADCC NK killing assay. 50 μL of antibody were added to the TILs, followed by allogeneic primary NK cells at an E/T ratio of 20:1.

NK cells from healthy donors were previously expanded using Miltenyi NK isolation and expansion kit. Aliquots were thawed and stored at 37° C. in TIL media at 10⁷ cell/mL until TILs preparation. The amount of NK cells needed for the experiment was calculated based on the number of target cells (the Tregs) previously assessed during the phenotyping experiment. The final volume was adjusted at 200 mL before 24-hour co-culture at 37° C., 5% CO₂. FACS staining was performed to identify immune cell populations as described in Example 15. Samples were analyzed by flow cytometry using FlowJo software: B cells were gated as Alive+/CD45+/CD19+; CD8+ cells were gated as Alive+/CD45+/CD3+/CD8+; Teff cells were gated as Alive+/CD45+/CD3+/CD4+/FoxP3- and Tregs were gated as Alive+/CD45+/CD3+/CD4+/FoxP3+/TIGIT+. Statistical analysis was performed using one-way ANOVA test (**** p<0.0001, *** p<0.001, ** p<0.01, *p<0.05)

A strong Treg decrease (as % of CD3+) was observed compared to the isotype group (from 50% to 90%, mean of 71.3=13%, p value<0.0001) (FIG. 17B). This population disappeared in the dot plot of one representative patient in FIG. 17C. This depletion was specific to FoxP3+CD4+ T cells (Tregs) as there was no decrease observed on the other cell populations as shown in FIG. 17B. Conversely, a significant increase of CD8+ cells was observed (CD8+ of CD3+ cells) with a mean of +9.8=9%, ** p=0.0067). Furthermore, the B-cell population significantly increased in the treated group with +16.7±19.6% (p<0.0317). Anti-CCR8 antibody was able to modulate TME by specific Tregs depletion ex vivo and restore anti-tumor immunity by increasing CD8+ T-cells and B-cells in TILs from RCC patients.

As further illustrated in FIG. 17D, depletion of Tregs after overnight co-culture of primary tumor-infiltrating lymphocytes (TILs) with primary NK cells at an Effector:Target ratio of 20:1 was assessed in a dose response experiment. Remaining cells were quantified by flow cytometry. Depletion of Tregs in ADCC assay as a function of amount of exogenous NK cells added was also assessed (FIG. 17E). Two out of nine samples profiled showed substantial Treg depletion even in the absence of exogenous NK cells, suggesting that endogenous NK cells present in the tumor sample are able to mediate ADCC.

### Example 19: Safety and Pharmacokinetics Evaluation

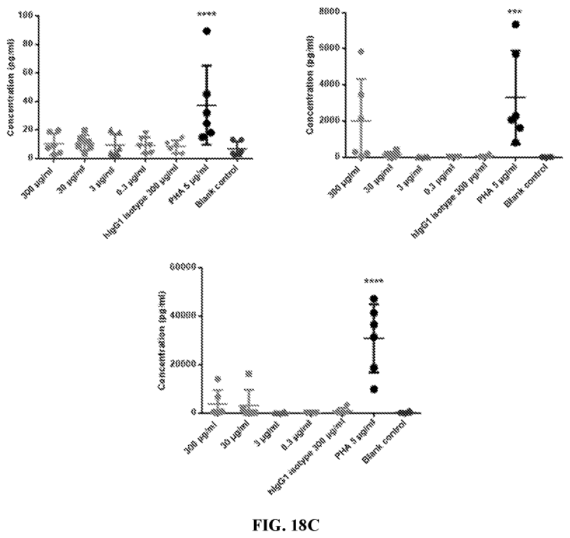

Pharmacokinetics and safety studies of HFB101110 were performed in cynomolgus monkeys (see FIG. 18A for a schematic of the single-dose PK study).

Serum PK profile of HFB101110 in cynomolgus monkeys revealed that no anti-drug antibodies were detected after 15 days (see FIG. 18B), and that in a in vitro cytokine release study from human PBMCs using a soluble antibody format, no significant cytokine release was observed (see FIG. 18C).

### REFERENCES

Villarreal, D. O., L'Huillier, A., Armington, S., Mottershead, C., Filippova, E. V., Coder, B. D., Petit, R. G. & Princiotta, M. F. (2018) Targeting CCR8 induces protective antitumor immunity and enhances vaccine-induced responses in colon cancer, Cancer Res. 78, 5340-5348.
Van Damme, H., Dombrecht, B., Kiss, M., Roose, H., Allen, E., Van Overmeire, E., Kancheva, D., Martens, L., Murgaski, A., Bardet, P. M. R., Blancke, G., Jans, M., Bolli, E., Martins, M. S., Elkrim, Y., Dooley, J., Boon, L., Schwarze, J. K., Tacke, F., Movahedi, K., Vandamme, N., Neyns, B., Ocak, S., Scheyltjens, I., Vereecke, L., Nana, F. A., Merchiers, P., Laoui, D. & Van Ginderachter, J. A. (2021) Therapeutic depletion of CCR8 (+) tumor-infiltrating regulatory T cells elicits antitumor immunity and synergizes with anti-PD-1 therapy, J Immunother Cancer. 9, c001749.
Campbell, J. R., McDonald, B. R., Mesko, P. B., Siemers, N. O., Singh, P. B., Selby, M., Sproul, T. W., Korman, A. J., Vlach, L. M., Houser, J., Sambanthamoorthy, S., Lu, K., Hatcher, S. V., Lohre, J., Jain, R. & Lan, R. Y. (2021) Fc-Optimized Anti-CCR8 Antibody Depletes Regulatory T Cells in Human Tumor Models, Cancer Res. 81, 2983-2994.
Whiteside, S. K., Grant, F. M., Gyori, D. S., Conti, A. G., Imianowski, C. J., Kuo, P., Nasrallah, R., Sadiyah, F., Lira, S. A., Tacke, F., Eil, R. L., Burton, O. T., Dooley, J., Liston, A., Okkenhaug, K., Yang, J. & Roychoudhuri, R. (2021) CCR8 marks highly suppressive Treg cells within tumours but is dispensable for their accumulation and suppressive function, Immunology. 163, 512-520.
Bhatt, D., Kang, B., Sawant, D., Zheng, L., Perez, K., Huang, Z., Sekirov, L., Wolak, D., Huang, J. Y., Liu, X., DeVoss, J., Manzanillo, P. S., Pierce, N., Zhang, Z., Symons, A. & Ouyang, W. (2021) STARTRAC analyses of scRNAseq data from tumor models reveal T cell dynamics and therapeutic targets, J Exp Med. 218. e20201329.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An isolated monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having a polypeptide sequences of:
(1) SEQ ID NOs: 1, 48, 49, 50, 51, and 6, respectively;
(2) SEQ ID NOs: 1, 2, 3, 4, 5, and 6, respectively;
(3) SEQ ID NOs: 13, 2, 14, 4, 28, and 6, respectively;
(4) SEQ ID NOs: 13, 2, 15, 4, 5, and 6, respectively;
(5) SEQ ID NOs: 16, 17, 18, 29, 30, and 6, respectively;
(6) SEQ ID NOs: 19, 20, 21, 4, 5, and 6, respectively;
(7) SEQ ID NOs: 22, 23, 24, 31, 5, and 32, respectively;
(8) SEQ ID NOs: 25, 26, 27, 33, 34, and 35, respectively;
(9) SEQ ID NOs: 1, 2, 49, 4, 5, and 6, respectively; or
(10) SEQ ID NOs: 1, 2, 49, 50, 51, and 6, respectively;
wherein the antibody or antigen-binding fragment thereof specifically binds chemokine (C—C motif) receptor 8 (CCR8), preferably human CCR8.

2. The isolated monoclonal antibody or antigen-binding fragment thereof of claim 1, comprising a heavy chain variable region having a polypeptide sequence at least 95% identical to SEQ ID NO: 9, 7, 36, 38, 40, 42, 44, 46, 52, 53, 54, 55, 56, 57, 58, 59, or 60 or a light chain variable region having a polypeptide sequence at least 95% identical to SEQ ID NO: 10, 8, 37, 39, 41, 43, 45, 47, 61, 62, or 63.

3. The isolated monoclonal antibody or antigen-binding fragment thereof of claim 1, comprising:
(1) a heavy chain variable region having the polypeptide sequence of SEQ ID NO: 9, and a light chain variable region having the polypeptide sequence of SEQ ID NO: 10;
(2) a heavy chain variable region having the polypeptide sequence of SEQ ID NO: 7, and a light chain variable region having the polypeptide sequence of SEQ ID NO: 8;
(3) a heavy chain variable region having the polypeptide sequence of SEQ ID NO: 36, and a light chain variable region having the polypeptide sequence of SEQ ID NO: 37;
(4) a heavy chain variable region having the polypeptide sequence of SEQ ID NO: 38, and a light chain variable region having the polypeptide sequence of SEQ ID NO: 39;
(5) a heavy chain variable region having the polypeptide sequence of SEQ ID NO: 40, and a light chain variable region having the polypeptide sequence of SEQ ID NO: 41;
(6) a heavy chain variable region having the polypeptide sequence of SEQ ID NO: 42, and a light chain variable region having the polypeptide sequence of SEQ ID NO: 43;
(7) a heavy chain variable region having the polypeptide sequence of SEQ ID NO: 44, and a light chain variable region having the polypeptide sequence of SEQ ID NO: 45;
(8) a heavy chain variable region having the polypeptide sequence of SEQ ID NO: 46, and a light chain variable region having the polypeptide sequence of SEQ ID NO: 47;
(9) a heavy chain variable region having the polypeptide sequence of SEQ ID NO: 52, and a light chain variable region having the polypeptide sequence of SEQ ID NO: 63;
(10) a heavy chain variable region having the polypeptide sequence of SEQ ID NO: 52, and a light chain variable region having the polypeptide sequence of SEQ ID NO: 10;
(11) a heavy chain variable region having the polypeptide sequence of SEQ ID NO: 53, and a light chain variable region having the polypeptide sequence of SEQ ID NO: 10;
(12) a heavy chain variable region having the polypeptide sequence of SEQ ID NO: 54 and a light chain variable region having the polypeptide sequence of SEQ ID NO: 63; or
(13) a heavy chain variable region having the polypeptide sequence of SEQ ID NO: 59 and a light chain variable region having the polypeptide sequence of SEQ ID NO: 62.

4.-7. (canceled)

8. The isolated monoclonal antibody or antigen-binding fragment thereof of claim 1, wherein the antibody or antigen-binding fragment thereof is capable of inducing effector-mediated tumor cell lysis through antibody-dependent cellular cytotoxicity (ADCC); antibody-dependent cellular phagocytosis (ADCP); and/or mediating recruitment of conjugated drugs; and/or forming a bispecific antibody with another mAb or antigen-binding fragment thereof with cancer-killing effect.

9. The isolated monoclonal antibody or antigen-binding fragment thereof of claim 1, wherein the monoclonal antibody or antigen-binding fragment thereof specifically binds cynomolgus CCR8.

10. A bispecific antibody or antigen-binding fragment thereof comprising the monoclonal antibody or antigen-binding fragment thereof of claim 1.

11. An isolated nucleic acid encoding the monoclonal antibody or antigen-binding fragment thereof of claim 1.

12. An isolated nucleic acid encoding the bispecific antibody or antigen-binding fragment thereof of claim 10.

13. A vector comprising the isolated nucleic acid of claim 12.

14. A host cell comprising the vector of claim 13.

15. A pharmaceutical composition, comprising the isolated monoclonal antibody or antigen-binding fragment thereof of claim 1 and a pharmaceutically acceptable carrier.

16. A method of targeting CCR8 on a cancer cell surface, and/or treating a cancer in a subject in need thereof, comprising administering to the subject the pharmaceutical composition of claim 15.

17. The method of claim 16, wherein the cancer is a solid tumor, preferably a solid tumor with infiltrating T cells, more preferably a solid tumor with infiltrating T reg cells, more preferably a solid tumor with highly suppressive T reg cells expressing CCR8, most preferably a solid tumor with infiltrating highly suppressive T reg cells overexpressing CCR8 for which natural killer (NK) cell infiltration has occurred.

18. The method of claim 16, wherein the cancer is selected from the group consisting of lung cancer, head and neck cancer, esophageal cancer, stomach cancer, colorectal cancer, breast cancer, pancreatic cancer, ovarian cancer, kidney cancer, and melanoma.

19. The method of claim 16, wherein the subject comprises CCR8-expressing Treg cells.

20. A method of producing the monoclonal antibody or antigen-binding fragment thereof of claim 1, comprising culturing a cell comprising a nucleic acid encoding the monoclonal antibody or antigen-binding fragment thereof under conditions to produce the monoclonal antibody or antigen-binding fragment thereof and recovering the monoclonal antibody or antigen-binding fragment thereof from the cell or culture.

21. A method of producing a pharmaceutical composition comprising the monoclonal antibody or antigen-binding fragment of claim 1, comprising combining the monoclonal antibody or antigen-binding fragment thereof with a pharmaceutically acceptable carrier to obtain the pharmaceutical composition.

22. A method of determining a level of CCR8 in a subject, the method comprising:
a. obtaining a sample from the subject;
b. contacting the sample with an isolated monoclonal antibody or antigen-binding fragment thereof of claim 1; and
c. determining the level of CCR8 in the subject.

23. The method of claim 22, wherein the sample is a tissue sample or a blood sample, optionally wherein the tissue sample is a cancer tissue sample.

24. The method of claim 22, wherein the sample comprises Treg cells.

25. An isolated monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having a polypeptide sequences of SEQ ID NOs: 1, 48, 49, 50, 51, and 6, respectively.

26. The isolated monoclonal antibody or antigen-binding fragment thereof of claim 25 comprising a heavy chain variable region having a polypeptide sequence of SEQ ID NO: 9 and a light chain variable region having a polypeptide sequence of SEQ ID NO: 10.

27. An isolated monoclonal antibody or antigen-binding fragment thereof comprising a heavy chain variable region having a polypeptide sequence at least 95% identical to SEQ ID NO: 9 and a light chain variable region having a polypeptide sequence at least 95% identical to SEQ ID NO: 10.

28. A pharmaceutical composition, comprising the bispecific antibody or antigen-binding fragment thereof of claim 10 and a pharmaceutically acceptable carrier.

29. A method of targeting CCR8 on a cancer cell surface, and/or treating a cancer in a subject in need thereof, comprising administering to the subject the pharmaceutical composition of claim 28.

30. The method of claim 29, wherein the cancer is a solid tumor, preferably a solid tumor with infiltrating T cells, more preferably a solid tumor with infiltrating T reg cells, more preferably a solid tumor with highly suppressive T reg cells expressing CCR8, most preferably a solid tumor with infiltrating highly suppressive T reg cells overexpressing CCR8 for which natural killer (NK) cell infiltration has occurred.

31. The method of claim 29, wherein the cancer is selected from the group consisting of lung cancer, head and neck cancer, esophageal cancer, stomach cancer, colorectal cancer, breast cancer, pancreatic cancer, ovarian cancer, kidney cancer, and melanoma.

32. The method of claim 29, wherein the subject comprises CCR8-expressing Treg cells.

33. A method of producing the bispecific antibody or antigen-binding fragment thereof of claim 10, comprising culturing a cell comprising a nucleic acid encoding the bispecific antibody or antigen-binding fragment thereof under conditions to produce the bispecific antibody or antigen-binding fragment thereof and recovering the bispecific antibody or antigen-binding fragment thereof from the cell or culture.

34. A method of producing a pharmaceutical composition comprising the bispecific antibody or antigen-binding fragment thereof of claim 10, comprising combining the bispecific antibody or antigen-binding fragment thereof with a pharmaceutically acceptable carrier to obtain the pharmaceutical composition.


SEQUENCE LISTING (65 sequences)

SEQ ID NO: 1 - protein, 8 aa, synthetic construct
  REGION 1..8: note = HFB11-10 HCDR1
GFRFNTNA

SEQ ID NO: 2 - protein, 11 aa, synthetic construct
  REGION 1..11: note = HFB11-10 HCDR2
IRSKSNNYATY

SEQ ID NO: 3 - protein, 14 aa, synthetic construct
  REGION 1..14: note = HFB11-10 HCDR3
VRGSDNNYYYAMDY

SEQ ID NO: 4 - protein, 11 aa, synthetic construct
  REGION 1..11: note = HFB11-10 LCDR1
KSLLHSNGNTY

SEQ ID NO: 5 - protein, 6 aa, synthetic construct
  REGION 1..6: note = HFB11-10 LCDR2
YRMSNL

SEQ ID NO: 6 - protein, 9 aa, synthetic construct
  REGION 1..9: note = HFB11-10 LCDR3
MQHLEYPFT

SEQ ID NO: 7 - protein, 123 aa, synthetic construct
  REGION 1..123: note = HFB11-10 VH
EVQLVETGGGLVQPKGSLKLSCAASGFRFNTNAMNWVRQAPGKDLEWVARIRSKSNNYATYYADSVKDRFTISRDDSQSMLYLQMNNVKTEDTAMYYCVRGSDNNYYYAMDYWGQGTSVTVSS

SEQ ID NO: 8 - protein, 112 aa, synthetic construct
  REGION 1..112: note = HFB11-10 VL
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 9 - protein, 123 aa, synthetic construct
  REGION 1..123: note = HFB11-10 Humanized VH
EVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSDNNYYYAMDYWGQGTTVTVSS

SEQ ID NO: 10 - protein, 112 aa, synthetic construct
  REGION 1..112: note = HFB11-10 Humanized VL
DIVMTQSPLSLPVTPGEPASISCRSSQSLLHSNGNTYLYWFLQKPGQSPQLLIYRMSNRASGVPDRFSGSGSGTDFTLKISRVEAEDVGVYYCMQHLEYPFTFGQGTKLEIK

SEQ ID NO: 11 - protein, 355 aa, synthetic construct
  REGION 1..355: note = Human CCR8 peptide
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 12 - protein, 360 aa, synthetic construct
  REGION 1..360: note = Human CCR4 peptide
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 13 - protein, 8 aa, synthetic construct
  REGION 1..8: note = HFB11-2 HCDR1
GFSFNTYA

SEQ ID NO: 14 - protein, 14 aa, synthetic construct
  REGION 1..14: note = HFB11-2 HCDR3
VRGGSYYSNHYFDY

SEQ ID NO: 15 - protein, 14 aa, synthetic construct
  REGION 1..14: note = HFB11-3 HCDR3
VRGKDANYFYAMDY

SEQ ID NO: 16 - protein, 8 aa, synthetic construct
  REGION 1..8: note = HFB11-5 HCDR1
GFSFNAYA

SEQ ID NO: 17 - protein, 11 aa, synthetic construct
  REGION 1..11: note = HFB11-5 HCDR2
IRTKSNNYATY

SEQ ID NO: 18 - protein, 14 aa, synthetic construct
  REGION 1..14: note = HFB11-5 HCDR3
VRGGYGNGGYYFDY

SEQ ID NO: 19 - protein, 8 aa, synthetic construct
  REGION 1..8: note = HFB11-8 HCDR1
GFRFNTNA

SEQ ID NO: 20 - protein, 11 aa, synthetic construct
  REGION 1..11: note = HFB11-8 HCDR2
IRSKSNNYATH

SEQ ID NO: 21 - protein, 14 aa, synthetic construct
  REGION 1..14: note = HFB11-8 HCDR3
VRGSDNNYYYAMDY

SEQ ID NO: 22 - protein, 8 aa, synthetic construct
  REGION 1..8: note = HFB11-19 HCDR1
GYTFTDYI

SEQ ID NO: 23 - protein, 9 aa, synthetic construct
  REGION 1..9: note = HFB11-19 HCDR2
FSPRNNYVK

SEQ ID NO: 24 - protein, 12 aa, synthetic construct
  REGION 1..12: note = HFB11-19 HCDR3
SRHGRNSIGFAY

SEQ ID NO: 25 - protein, 8 aa, synthetic construct
  REGION 1..8: note = HFB11-21 HCDR1
GYTFISYW

SEQ ID NO: 26 - protein, 9 aa, synthetic construct
  REGION 1..9: note = HFB11-21 HCDR2
IDPNSGKTK

SEQ ID NO: 27 - protein, 8 aa, synthetic construct
  REGION 1..8: note = HFB11-21 HCDR3
AREGWGDY

SEQ ID NO: 28 - protein, 6 aa, synthetic construct
  REGION 1..6: note = HFB11-2 LCDR2
YQMSNL

SEQ ID NO: 29 - protein, 11 aa, synthetic construct
  REGION 1..11: note = HFB11-5 LCDR1
KSLLHRNGNSY

SEQ ID NO: 30 - protein, 6 aa, synthetic construct
  REGION 1..6: note = HFB11-5 LCDR2
YRISNL

SEQ ID NO: 31 - protein, 11 aa, synthetic construct
  REGION 1..11: note = HFB11-19 LCDR1
KSLLNSNGNTY

SEQ ID NO: 32 - protein, 9 aa, synthetic construct
  REGION 1..9: note = HFB11-19 LCDR3
MQHLEYPYT

SEQ ID NO: 33 - protein, 10 aa, synthetic construct
  REGION 1..10: note = HFB11-21 LCDR1
ESVDNYGISF

SEQ ID NO: 34 - protein, 6 aa, synthetic construct
  REGION 1..6: note = HFB11-21 LCDR2
YRASNL

SEQ ID NO: 35 - protein, 9 aa, synthetic construct
  REGION 1..9: note = HFB11-21 LCDR3
QQSNKDPRT

SEQ ID NO: 36 - protein, 123 aa, synthetic construct
  REGION 1..123: note = HFB11-2 VH
EVQLVESGGGLVQPKGSLKLSCAASGFSFNTYAMNWVRQAPGKGLEWVARIRSKSNNYATYYADSVKDRFTISRDDSERMLYLQMNNLKTEDTAMYYCVRGGSYYSNHYFDYWGQGTTLTVSS

SEQ ID NO: 37 - protein, 112 aa, synthetic construct
  REGION 1..112: note = HFB11-2 VL
DIVMTQAAPSVPVTPGESVSISCRSSKSLLHSNGNTYLYWFLQRPGQSPQLLIYQMSNLASGVPDRFSGSGSGTAFTLRISRVEAEDVGVYYCMQHLEYPFTFGAGTKLELK

SEQ ID NO: 38 - protein, 123 aa, synthetic construct
  REGION 1..123: note = HFB11-3 VH
EVQLVESGGGLVQPQGSLKLSCAASGFSFNTYAMNWVRQAPGKGLQWVARIRSKSNNYATYYADSVKDRFTISRDDSESMLYLQMNNLKTEDAAMYYCVRGKDANYFYAMDYWGQGTSVTVSS

SEQ ID NO: 39 - protein, 112 aa, synthetic construct
  REGION 1..112: note = HFB11-3 VL
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGAGTKLELK

SEQ ID NO: 40 - protein, 123 aa, synthetic construct
  REGION 1..123: note = HFB11-5 VH
EVQLVESGGGLVQPRGSLKLSCTASGFSFNAYAMNWVRQAPGKGLEWVARIRTKSNNYATYYADSVKDRFTISRDDSESMLYLQMKNLKTEDTAMYYCVRGGYGNGGYYFDYWGQGTTLTVSS

SEQ ID NO: 41 - protein, 112 aa, synthetic construct
  REGION 1..112: note = HFB11-5 VL
DIVMTQAAPSVTVTPGESASISCRSSKSLLHRNGNSYLYWFLQRPGQSPQLLIYRISNLASGVPDRFSGSGSGTAFTLRISRVEAEDVGVYYCMQHLEYPFTFGSGTKLEIK

SEQ ID NO: 42 - protein, 123 aa, synthetic construct
  REGION 1..123: note = HFB11-8 VH
EVQLVETGGGLVQPKGSLKLSCAASGFRFNTNAMNWVRQAPGKGLEWVARIRSKSNNYATHYVDSVKDRFTVSRDDSQSMLYLQMNNLKAEDTAMYYCVRGSDNNYYYAMDYWGQGTSVTVSS

SEQ ID NO: 43 - protein, 112 aa, synthetic construct
  REGION 1..112: note = HFB11-8 VL
DIVMTQAAPSVPVTPGESVSISCRSSKSLLHSNGNTYLYWFLQRPGQSPQLLIYRMSNLASGVPDRFSGSGSGTSFTLRISRVEVEDVGVYYCMQHLEYPFTFGSGTKLEIK

SEQ ID NO: 44 - protein, 119 aa, synthetic construct
  REGION 1..119: note = HFB11-19 VH
QVQLQQSGAELVKPGASVKLSCKASGYTFTDYIIHWVKQRSGQGLEWIGWFSPRNNYVKYNERFTDKATLTADKSSNTVYMELSSLTSEDSAVYFCSRHGRNSIGFAYWGQGTLVTVSA

SEQ ID NO: 45 - protein, 112 aa, synthetic construct
  REGION 1..112: note = HFB11-19 VL
DIVMTQAAPSVPVTPGESVSISCRSSKSLLNSNGNTYLYWFLQRPGQSPQLLIYRMSNLASGVPDRFSGSGSGTAFTLRISRVEAEDVGVYYCMQHLEYPYTFGGGTKLEIK

SEQ ID NO: 46 - protein, 115 aa, synthetic construct
  REGION 1..115: note = HFB11-21 VH
QVQLQQPGAELVKPGASVKLSCKASGYTFISYWMHWVKQRPGRGLEWIGRIDPNSGKTKYNEKFKTKATLTVDKSSSTAYMHLNSLTSDDSAVYYCAREGWGDYWGQGTTLTVSS

SEQ ID NO: 47 - protein, 111 aa, synthetic construct
  REGION 1..111: note = HFB11-21 VL
DIVLTQSPASLAVSLGQRATISCRASESVDNYGISFMHWYQQKPGQPPRLLIYRASNLASGIPARFSGSGSSTDFTLTINPVETDDIATYYCQQSNKDPRTFGGGTKLEIK

SEQ ID NO: 48 - protein, 11 aa, synthetic construct
  REGION 1..11: note = H11-10 VH10 HCDR2
IRSKSNSYATY

SEQ ID NO: 49 - protein, 14 aa, synthetic construct
  REGION 1..14: note = H11-10 VH10 HCDR3
TRGSDNNYYYAMDY

SEQ ID NO: 50 - protein, 11 aa, synthetic construct
  REGION 1..11: note = H11-10 VL1 LCDR1
QSLLHSNGNTY

SEQ ID NO: 51 - protein, 6 aa, synthetic construct
  REGION 1..6: note = H11-10 VL1 LCDR2
YRMSNR

SEQ ID NO: 52 - protein, 123 aa, synthetic construct
  REGION 1..123: note = H11-10 VH1
EVQLVESGGGLVQPGGSLKLSCAASGFRFNTNAMNWVRQASGKGLEWVGRIRSKSNNYATYYAASVKGRFTISRDDSKNTAYLQMNSLKTEDTAVYYCTRGSDNNYYYAMDYWGQGTTVTVSS

SEQ ID NO: 53 - protein, 123 aa, synthetic construct
  REGION 1..123: note = H11-10 VH2
EVQLVESGGGLVQPGGSLKLSCAASGFRFNTNAMNWVRQASGKDLEWVARIRSKSNNYATYYAASVKGRFTISRDDSKNTLYLQMNSLKTEDTAVYYCTRGSDNNYYYAMDYWGQGTTVTVSS

SEQ ID NO: 54 - protein, 123 aa, synthetic construct
  REGION 1..123: note = H11-10 VH3
QVQLVESGGGVVQPGRSLRLSCAASGFRFNTNAMNWVRQAPGKGLEWVARIRSKSNNYATYYADSVKGRFTISRDNSKNTLYLQMNSLRAEDTAVYYCVRGSDNNYYYAMDYWGQGTTVTVSS

SEQ ID NO: 55 - protein, 123 aa, synthetic construct
  REGION 1..123: note = H11-10 VH4
EVQLVESGGGLVQPGRSLRLSCTASGFRFNTNAMNWFRQAPGKGLEWVARIRSKSNNYATYYTASVKGRFTISRDGSKSIAYLQMNSLKTEDTAVYYCVRGSDNNYYYAMDYWGQGTTVTVSS

SEQ ID NO: 56 - protein, 123 aa, synthetic construct
  REGION 1..123: note = H11-10 VH5
QVQLVQSGAEVKKPGASVKVSCKASGFRFNTNAMNWVRQAPGQDLEWVARIRSKSNNYATYYSQKFQGRVTITRDTSASTAYMELSSLRSEDTAVYYCVRGSDNNYYYAMDYWGQGTTVTVSS

SEQ ID NO: 57 - protein, 123 aa, synthetic construct
  REGION 1..123: note = H11-10 VH6
QVQLQESGPGLVKPSGTLSLTCAASGFRFNTNAMNWVRQPPGKGLEWVGRIRSKSNNYATYYNPSLKSRVTISRDKSKNQFSLKLSSVTAADTAVYYCVRGSDNNYYYAMDYWGQGTTVTVSS

SEQ ID NO: 58 - protein, 123 aa, synthetic construct
  REGION 1..123: note = H11-10 VH8
QVTLRESGPALVKPTQTLTLTCTASGFRFNTNAMNWVRQPPGKALEWVARIRSKSNNYATYYSTSVKTRLTISKDTSKNQLVLTMTNMDPVDTATYYCVRGSDNNYYYAMDYWGQGTTVTVSS

SEQ ID NO: 59 - protein, 123 aa, synthetic construct
  REGION 1..123: note = H11-10 VH9
EVQLVESGGGLVQPGGSLKLSCAASGFRFNTNAMNWVRQASGKGLEWVGRIRSKSNSYATYYAASVKGRFTISRDDSKNTAYLQMNSLKTEDTAVYYCTRGSDNNYYYAMDYWGQGTTVTVSS

SEQ ID NO: 60 - protein, 123 aa, synthetic construct
  REGION 1..123: note = H11-10 VH11
QVQLVESGGGVVQPGGSLRLSCAASGFRFNTNAMNWVRQAPGKDLEWVARIRSKSNNYATYYADSVKGRFTISRDNSKNTLYLQMNSLRAEDTAVYYCVRGSDNNYYYAMDYWGQGTTVTVSS

SEQ ID NO: 61 - protein, 112 aa, synthetic construct
  REGION 1..112: note = H11-10 VL2
EIVMTQTPLSLSVTPGEQASISCRSSKSLLHSNGNTYLYWFLQKARPSSTLLIYRMSNLASGVPDRFSGSGSGTDFTLKISRVEAEDVGVYYCMQHLEYPFTFGQGTKLEIK

SEQ ID NO: 62 - protein, 112 aa, synthetic construct
  REGION 1..112: note = H11-10 VL3
DIQMTQSPSAMSVSVGDRVTITCRSSKSLLHSNGNTYLYWFQQKPGKVPKLLIYRMSNLASGVPSRFSGSGSGTEFTLTISSVQPEDVATYYCMQHLEYPFTFGQGTKLEIK

SEQ ID NO: 63 - protein, 112 aa, synthetic construct
  REGION 1..112: note = H11-10 VL4
EIVLTQSPGTLSLSPGERATLSCRSSKSLLHSNGNTYLYWFQQKPGQAPRLLIYRMSNLASGIPDRFSGSGSGTDFTLTISRLEPEDVAVYYCMQHLEYPFTFGQGTKLEIK

SEQ ID NO: 64 - protein, 353 aa, Mus musculus
MDYTMEPNVTMTDYYPDFFTAPCDAEFLLRGSMLYLAILYCVLFVLGLLGNSLVILVLVGCKKLRSITDIYLLNLAASDLLFVLSIPFQTHNLLDQWVFGTAMCKVVSGLYYIGFFSSMFFITLMSVDRYLAIVHAVYAIKVRTASVGTALSLTVWLAAVTATIPLMVFYQVASEDGMLQCFQFYEEQSLRWKLFTHFEINALGLLLPFAILLFCYVRILQQLRGCLNHNRTRAIKLVLTVVIVSLLFWVPFNVALFLTSLHDLHILDGCATRQRLALAIHVTEVISFTHCCVNPVIYAFIGEKFKKHLMDVFQKSCSHIFLYLGRQMPVGALERQLSSNQRSSHSSTLDDIL

SEQ ID NO: 65 - protein, 355 aa, Macaca fascicularis
MDYTLDPSMTTMTDYYYPDSLSSPCDGELIQRNDKLLLAVFYCLLFVFSLLGNSLVILVLVVCKKLRNITDIYLLNLALSDLLFVFSFPFQTYYQLDQWVFGTVMCKVVSGFYYIGFYSSMFFITLMSVDRYLAVVHAVYAIKVRTIRMGTTLSLVVWLTAIMATIPLLVFYQVASEDGVLQCYSFYNQQTLKWKIFTNFEMNILGLLIPFTIFMFCYIKILHQLKRCQNHNKTKAIRLVLIVVIASLLFWVPFNVVLFLTSLHSMHILDGCSISQQLNYATHVTEIISFTHCCVNPVIYAFVGEKFKKHLSEIFQKSCSHIFIYLGRQMPRESCEKSSSCQQHSFRSSSIDYIL